(19) United States
(12) Patent Application Publication
(10) Pub. No.: US 2004/0258661 A1
(43) Pub. Date: Dec. 23, 2004

(54) Generation of use of tc1 and tc2 cells

(76) Inventors: Daniel Fowler, Bethesda, MD (US); Unsu Jung, Ashburn, VA (US); Jeffrey A. Medin, North York (CA); Ronald E. Gress, Gaithersburg, MD (US); Andreas Erdmann, Washington, DC (US); Bruce Levine, Cherry Hill, NJ (US); Carl June, Merion Station, PA (US)

Correspondence Address: KLARQUIST SPARKMAN, LLP, 121 SW SALMON STREET, SUITE 1600, PORTLAND, OR, 97204, US

(21) Appl. No.: 10/494,540

(22) PCT Filed: Oct. 31, 2002
(86) PCT No.: PCT/US02/35240

Related U.S. Application Data
(60) Provisional application No. 60/336,473, filed on Oct. 31, 2001.

(51) Int. Cl. A61K 45/00; A61K 38/20

(52) U.S. Cl. 424/85.2; 424/93.71

(57) ABSTRACT

A method is provided for producing a population of CD8⁺ Tc1 and/or Tc2 lymphocytes ex vivo. The method includes stimulating a population of T cells obtained from a subject by contacting the population with an anti-CD3 monoclonal antibody and an antibody that specifically binds to a T cell costimulatory molecule in the presence of a Tc1 or Tc2 supportive environment to form a stimulated population of T cells. The stimulated population of CD8⁺ T cells is allowed to proliferate in a Tc1 or Tc2 supportive environment. Purified populations of Tc1 and Tc2 cells are disclosed herein, as are methods for their use.

### CROSS REFERENCE TO RELATED APPLICATION

This application claims priority to U.S. Provisional Application No. 60/336,473 filed Oct. 31, 2001.

### FIELD

This application relates to the methods for generation of CD8⁺ Tc1 and Tc2 cells, to purified populations of CD8⁺ Tc1 and Tc2 cells, and to therapeutic uses of purified CD8⁺ Tc1 and Tc2 cells.

### BACKGROUND

The T lymphocyte (“T cell”) is a key cell type in the human cellular immune system, providing both function and biochemical control. T cells are classified based on which cell surface receptors and cytokines they express. The expression of cell surface receptors CD4 and/or CD8 are generally used to define two broad classes of T cells; these cell surface receptors are involved in recognizing antigens presented to the T cells by antigen presenting cells (APC). Certain mature T cells express only CD4 but not CD8 (termed CD4⁺ cells), while other mature T cells express CD8 but not CD4 (termed CD8⁺ cells).

CD8⁺ cells recognize peptide antigens that are presented on MHC class I molecules. Upon activation by an APC (which involves binding of both a stimulatory antigen and a costimulatory ligand), a CD8⁺ T cell matures into a cytotoxic T cell, which has defined functions and characteristics. CD4⁺ T cells recognize antigens that are presented on MHC class II molecules. CD8⁺ T cells can differentiate into cytotoxic T cells (Tc). Tc cells have been divided into subclasses based on their cytokine secretion profiles. Tc1 cells secrete a specific set of cytokines, including interferon-γ (IFN-γ), interleukin-12 (IL-12), interleukin-2 (IL-2), interferon-γ, and lymphotoxin. Tc2 cells secrete different cytokines (such as IL-4, IL-5 and IL-10).

The common method for generating CD8⁺ Tc1 and Tc2 cells is in vitro allostimulation (Halverson et al. Blood, 90:2089-96, 1997). In this method, purified CD8⁺ cells are co-cultured with irradiated bone marrow-derived stimulator cells from an unrelated donor. In addition, IL-12 and TGF-β (Tc1 culture) or IL-4 (Tc2 culture) is added. However, the disadvantage of this method is that the numbers of cells generated and the level of cytokine polarization are lower than desired. Because this decreases the efficacy of in vivo effects, and the usefulness of the allostimulated Tc1 and Tc2 cells for therapy. More recently, a method using immobilized anti-CD3 and anti-CD28 monoclonal antibodies has been used to expand both human CD4⁺ and CD8⁺ cells (Garlie et al. J. Immunother. 22:336-45, 1999). However, this study did not disclose a method that could be used to shift the generated CD4⁺ and CD8⁺ cells towards a Th1, Th2, Tc1 or Tc2 phenotype. Therefore, there remains a need to develop a method which can generate therapeutically-effective numbers of Tc1 and Tc2 cells having the desired level of cytokine polarization, which can be used for immunotherapy.

Allogenic stem cell transplantation (alloSCT) is a potential curative treatment option for patients having hematologic and/or solid tumors. Donor T cells contained in the blood or marrow allograft mediate both beneficial and detrimental post-transplant immune effects. T cells mediate a potentially curative graft-versus-leukemia (GVL) effect and prevent marrow graft rejection, but also can generate graft-versus-host disease (GVHD). Therefore, the relative balance of these immune effects is currently a primary determinant of clinical outcome after allogeneic transplantation.

Clinical studies during the 1980's using T cell-depleted (TCD) marrow allografts clearly demonstrated the importance of T cell-mediated immune reactions after alloBMT. Recipients of TCD allografts had greatly reduced levels of GVHD, but had much higher rates of both graft rejection and leukemic relapse (Poynton, Bone Marrow Transplant. 3:265-279, 1988). Because TCD-alloBMT shifted the cause of mortality from GVHD to leukemia relapse and graft rejection, this approach did not represent a significant treatment advance relative to conventional T cell-replete alloBMT.

These observations have prompted investigation into the development of donor T cell administration methodologies which might preserve an anti-leukemic effect and prevent graft rejection while limiting GVHD. Such methods include the administration of only CD4⁺ donor T cells (Champlin et al., Transplant. Proc. 23:1695-6, 1991), or the delayed administration of donor T cells post-transplant (Kolb et al., Blood. 76:2462-5,1990). Both of these approaches have met with limited success, as leukemia relapse and significant levels of GVHD remain significant problems. Thus, there clearly is a need to identify and purify populations of T cells that can be used to prevent or limit the development of GVHD while mediating a GVL effect

### SUMMARY

Disclosed herein are novel methods for generating CD8⁺ Tc1 and Tc2 cells and the purification of these cells. Specifically, culture conditions are disclosed herein that allow Tc1 and Tc2 cells to be selectively propagated in vitro. The ability to grow and/or administer populations of Tc2 and/or Tc1 cells also represents a new therapy to mediate anti-tumor responses, such as enhancing graft-versus-tumor (GVT) effects, and prevent graft rejection due to reduce graft-versus-host disease (GVHD). Thus, the ability to generate Tc1 and Tc2 cells represents methods for improving allogeneic stem cell transplantation.

In one specific example, a method is provided for producing a population of substantially purified CD8⁺ Tc1 or Tc2 lymphocytes. The method includes stimulating a population of T cells, for example T cells which have been obtained or isolated from a subject, by contacting the population with an anti-CD3 monoclonal antibody and an antibody that specifically binds to a T cell costimulatory molecule in the presence of a Tc1 or Tc2 supportive environment to form a stimulated population of T cells. In one embodiment, the stimulated population of CD8⁺ T cells is allowed to proliferate in a Tc1 or Tc2 supportive environment.

Purified populations of Tc1 or Tc2 cells are disclosed herein, as are methods for their use in allogenic stem cell transplantation, for example for the treatment of hematologic or solid tumors or for establishing allogeneic chimerism with reduced GVHD for the purpose of permitting solid organ transplantation.

The foregoing and other objects, features, and advantages of the methods and cells described herein will become more apparent from the following detailed description of several embodiments, which proceeds with reference to the accompanying figures.

BRIEF DESCRIPTION OF THE FIGURES

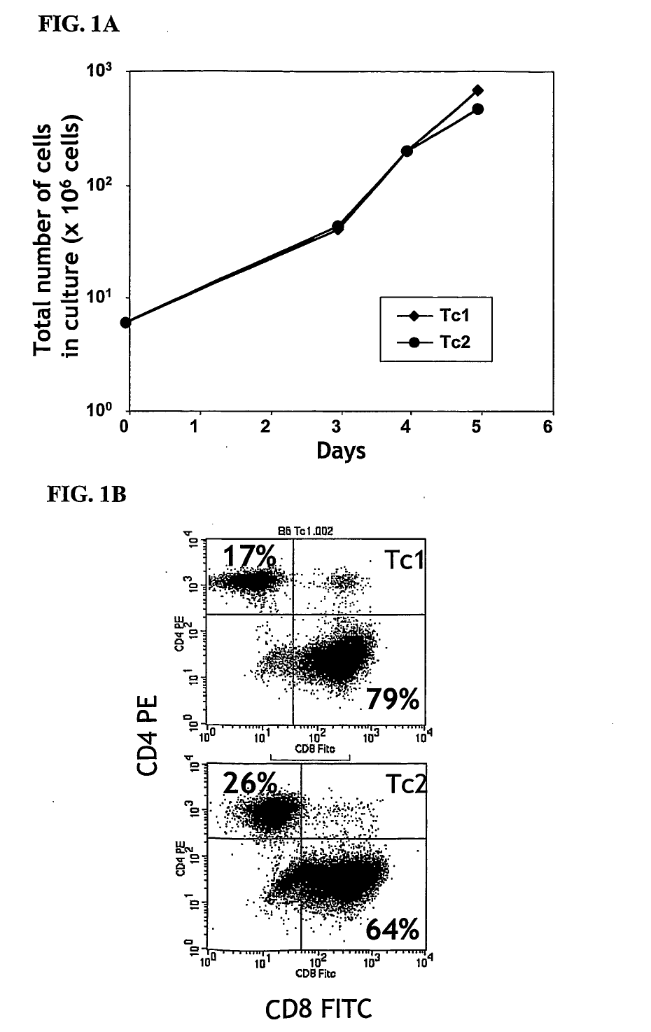

FIGS. 1A and 1B show a (A) line graph representation and a (B) matrix representation of the T cell yield of murine spleen cells cultured under conditions designed to induce Tc1 or Tc2 cell growth.

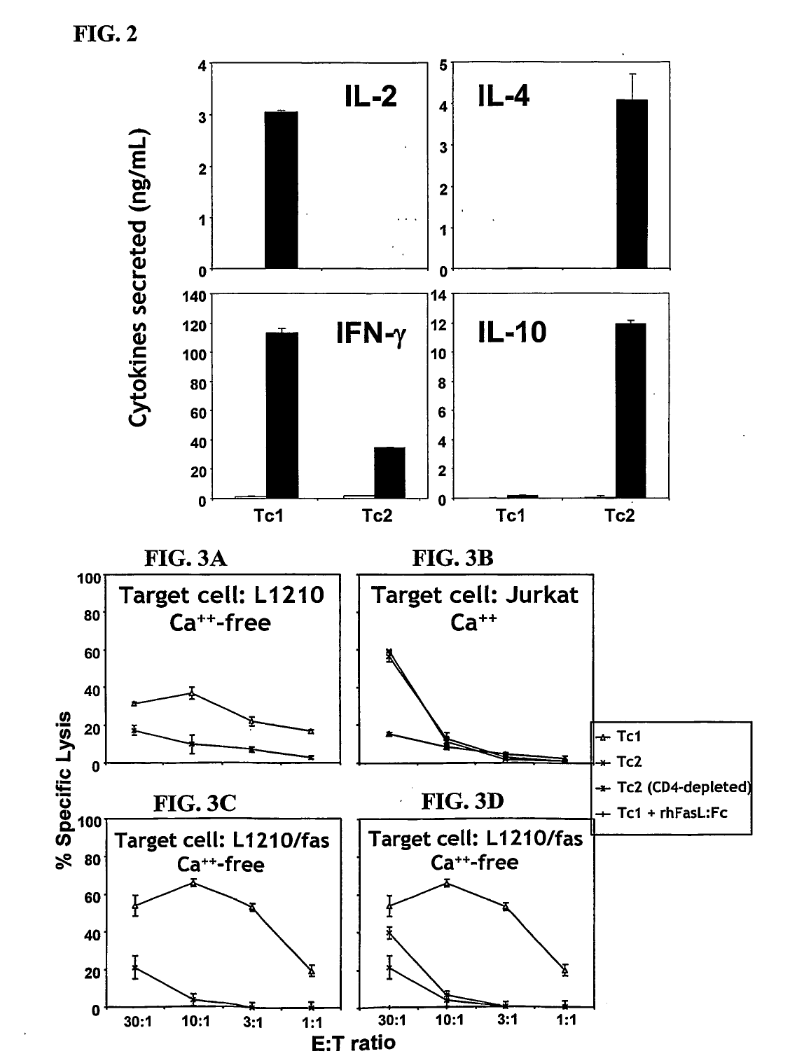

FIG. 2 shows four bar graphs showing the amount of cytokines produced when murine T cells were cultured under conditions designed to generate either Tc1 or Tc2 cells.

FIGS. 3A-D are graphs showing the percent of specific lysis by Tc1 or Tc2 cells under varying incubation conditions.

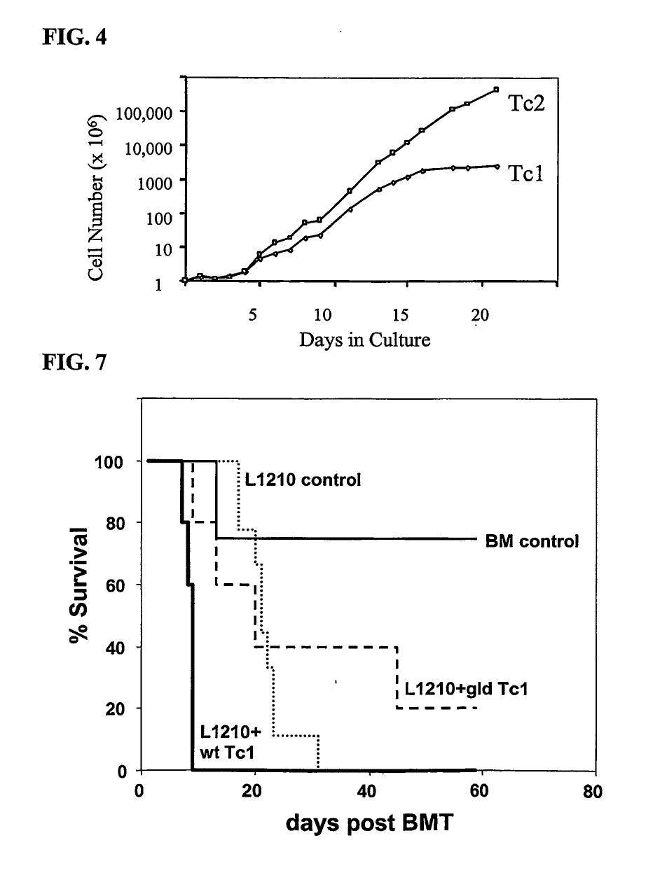

FIG. 4 is a graph showing the T cell yield of human lymphocytes cultured under conditions designed to induce Tc1 or Tc2 cell growth.

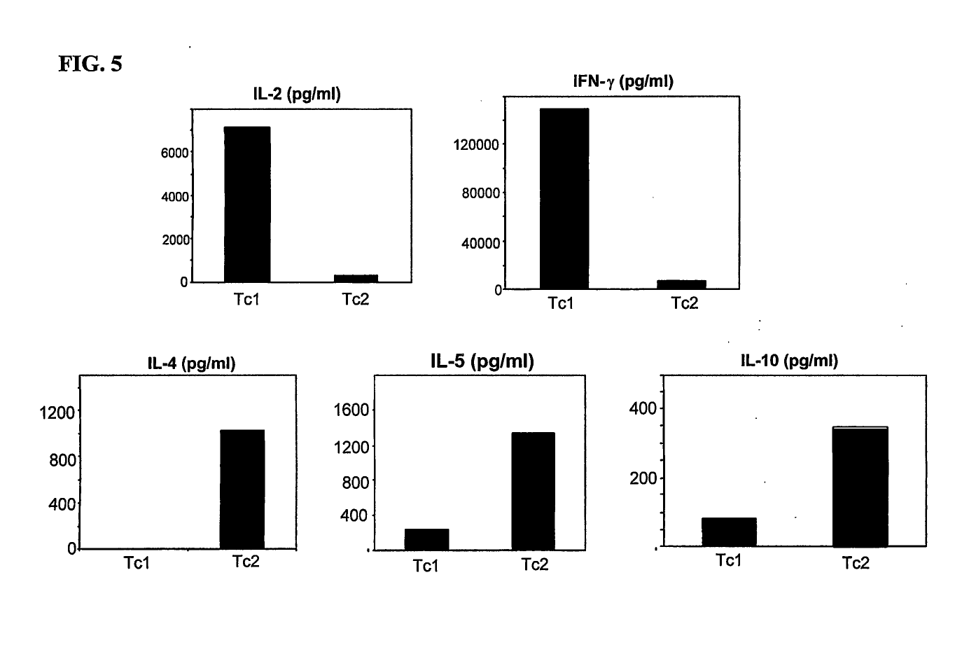

FIG. 5 shows five bar graphs showing the amount of cytolines produced when human T cells were cultured under conditions designed to generate either Tc1 or Tc2 cells.

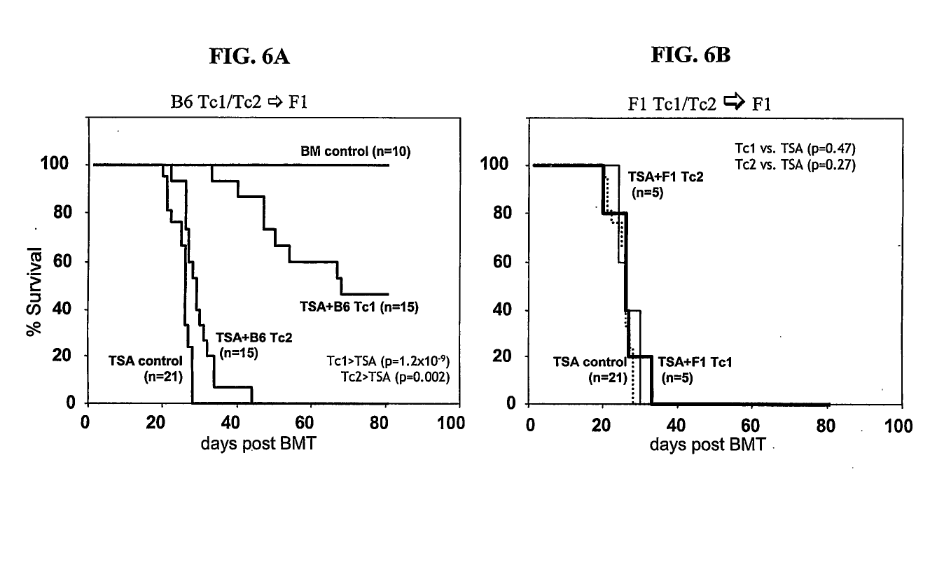

FIG. 6A is a graph showing the increase in survival time in mice receiving donor Tc1 or Tc2 cells of an allogenic background following a BMT and administration of cancer cells.

FIG. 6B is a graph showing that there is no increase in survival time in mice receiving donor Tc1 or Tc2 cells of a syngeneic background following a BMT and administration of cancer cells.

FIG. 7 is a graph showing that abrogating fas ligand function in Tc1 cells reduces GVHD associated with Tc1 cells, while retaining some GVL effect.

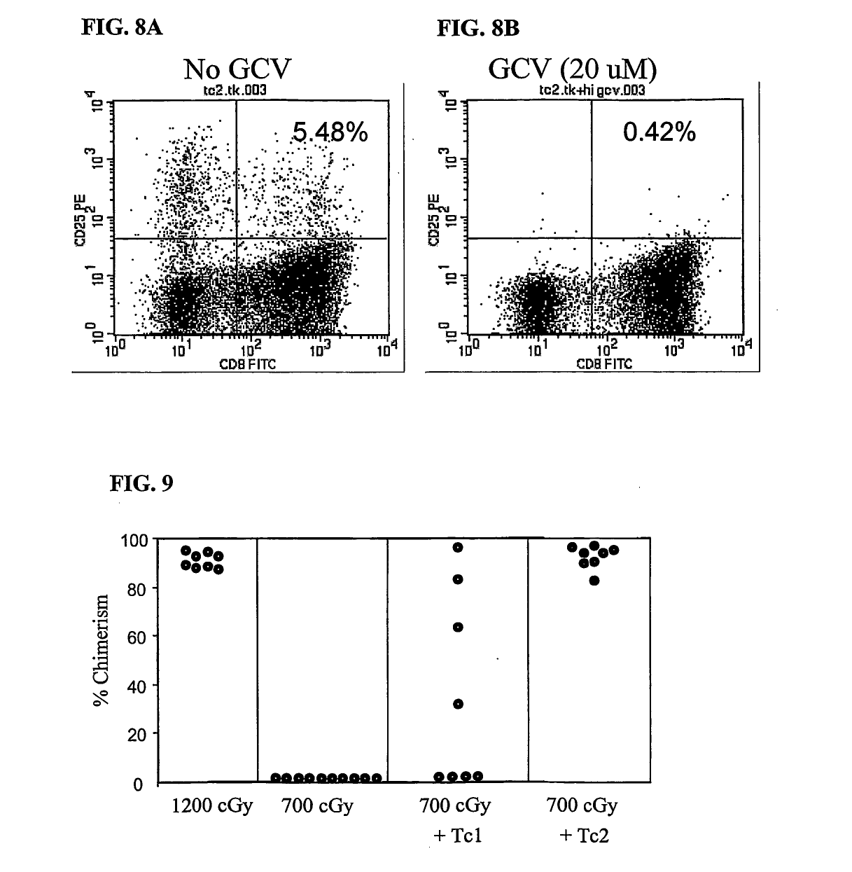

FIGS. 8A and 8B are flow cytometry plots showing the Tc2 population transduced with a suicide gene (A) before and (3) after treatment with ganciclovir (GCV), which is modulated into an effector metabolite by the suicide gene.

FIG. 9 is a dot-plot showing that administration of Tc1 cells partially abrogates marrow graft rejection while Tc2 cells abrogated marrow graft rejection.

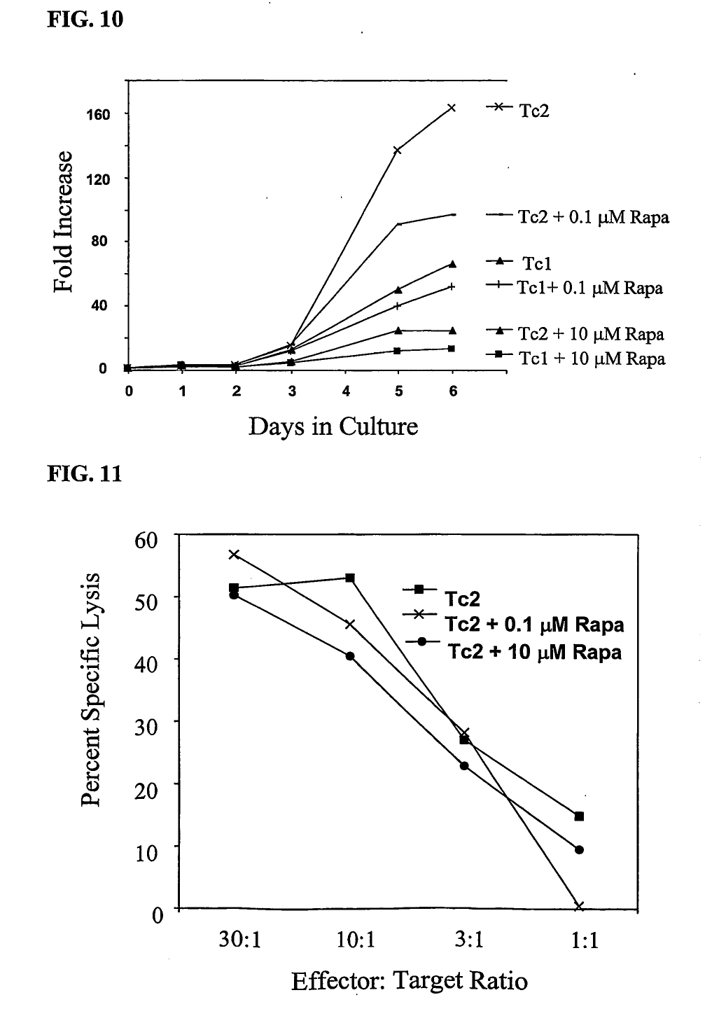

FIG. 10 is a graph showing that rapamycin reduces CD8⁺ T-cell expansion over the culture period under both Tc1 and Tc2 conditions.

FIG. 11 is a graph showing that rapamycin does not reduce or inhibit the cytotoxic capacity of Tc2 cells.

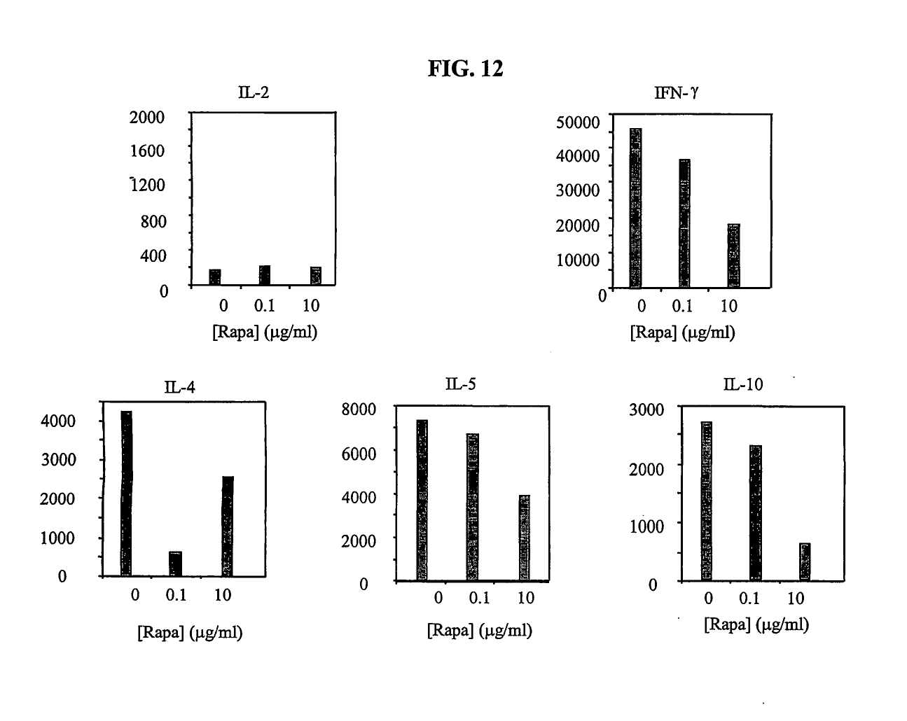

FIG. 12 shows five bar graphs showing the amount of cytolines produced when murine T cells were cultured under conditions designed to generate Tc2 cells, in the presence (0.1 μM or 10.0 μM) or absence of rapamycin.

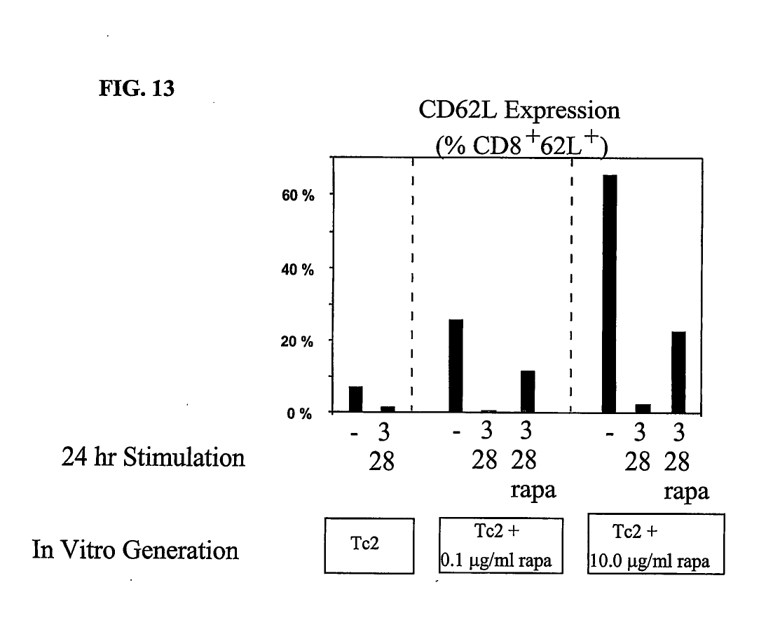

FIG. 13 is a bar graph showing that rapamycin increases Tc2 cell expression of L-selectin, CD62L.

### DETAILED DESCRIPTION OF SEVERAL EMBODIMENTS

### Abbreviations and Terms

The following explanations of terms and methods are provided to better describe the present disclosure and to guide those of ordinary skill in the art in the practice of the present disclosure. As used herein and in the appended claims, the singular forms “a” or “an” or “the” include plural references unless the context clearly dictates otherwise. For example, reference to “a cytokine” includes a plurality of such cytokines and reference to “the antibody” includes reference to one or more antibodies and equivalents thereof known to those skilled in the art, and so forth.

Unless explained otherwise, all technical and scientific terms used herein have the same meaning as commonly understood to one of ordinary skill in the art to which this disclosure belongs.

Animal: Living multicellular vertebrate organisms, a category which includes, for example, mammals and birds.

Antibody: Immunoglobulin molecules and immunologically active portions of immunoglobulin molecules, i.e., molecules that contain an antigen binding site which specifically binds (immunoreacts with) an antigen. In one embodiment the antigen is CD3. In another embodiment, the antigen is a co-stimulatory molecule (e.g. CD28).

A naturally occurring antibody (e.g., IgG) includes four polypeptide chains, two heavy (H) chains and two light (L) chains inter-connected by disulfide bonds. However, it has been shown that the antigen-binding function of an antibody can be performed by fragments of a naturally occurring antibody. Thus, these antigen-binding fragments are also intended to be designated by the term “antibody.” Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544-546, 1989) which consists of a VH domain; (v) an isolated complimentarity determining region (CDR); and (vi) an F(ab′)₂ fragment, a bivalent fragment comprising two Fab fragments linked by a disulfide bridge at the hinge region.

Furthermore, although the two domains of the Fv fragment are coded for by separate genes, a synthetic linker can be made that enables them to be made as a single protein chain (known as single chain Fv (scFv); Bird et al. Science 242:423-6, 1988; and Huston et al. Proc. Natl. Acad. Sci. 85:5879-83, 1988) by recombinant methods. Such single chain antibodies are also included. In one embodiment, antibody fragments for use in T cell expansion are those which are capable of crosslinking their target antigen, e.g., bivalent fragments such as F(ab′)₂ fragments. Alternatively, an antibody fragment which does not itself crosslink its target antigen (e.g., a Fab fragment) can be used in conjunction with a secondary antibody which serves to crosslink the antibody fragment, thereby crosslinking the target antigen. Antibodies can be fragmented using conventional techniques and the fragments screened for utility in the same manner as described for whole antibodies. An antibody is further intended to include bispecific and chimeric molecules that specifically bind the target antigen.

“Specifically binds” refers to the ability of individual antibodies to specifically immunoreact with an antigen, such as a T cell surface molecule. The binding is a non-random binding reaction between an antibody molecule and an antigenic determinant of the T cell surface molecule. The desired binding specificity is typically determined from the reference point of the ability of the antibody to differentially bind the T cell surface molecule and an unrelated antigen, and therefore distinguish between two different antigens, particularly where the two antigens have unique epitopes. An antibody that specifically binds to a particular epitope is referred to as a “specific antibody.”

Antigen: A substance capable of being the target of inducing a specific immune response.

B Cell: A lymphocyte, a type of white blood cell (leukocyte), that develops into a plasma cell, which produces antibodies.

Bone marrow transplant (BMT): The administration of bone marrow, for example intravenously, usually to an immuno-compromised subject whose immune cells have been depleted, for example by administration of cytotoxic agents. The marrow may be from a previously harvested and stored self-donation (autologous transplant), from a living donor other than the recipient (allogeneic transplant), or from an identical twin donor (syngeneic transplant). BMT is sometimes used to treat malignancies, such as leukemia, lymphoma, myeloma, and selected solid tumors, as well as nonmalignant conditions such as a plastic anemia, immunologic deficiencies, and inborn errors of metabolism.

Cancer: Malignant neoplasm that has undergone characteristic anaplasia with loss of differentiation, increase rate of growth, invasion of surrounding tissue, and is capable of metastasis.

Chemotherapy: In cancer treatment, chemotherapy refers to the administration of one or a combination of compounds to kill or slow the reproduction of rapidly multiplying cells. The amount of chemotherapeutic agent used for rheumatic or autoimmune conditions are usually lower than the doses used for cancer treatment. Chemotherapuetic agents include those known by those skilled in the art, including, but not limited to: 5-fluorouracil (5-FU), azathioprine, cyclophosphamide, antimetabolites (such as Fludarabine), antineoplastics (such as Etoposide, Doxorubicin, methotrexate, and Vincristine), carboplatin, cis-platinum and the taxanes, such as taxol.

Chemotherapy-resistant disease: A disorder that is not responsive to solely administration of a chemotherapeutic agent.

Comprises: A term that means “including.” For example, “comprising A or B” means including A or B, or both A and B, unless clearly indicated otherwise.

Costimulator of a T cell: Although stimulation of the TCR/CD3 complex (or CD2 molecule) is a primary activation signal in a T cell, a number of molecules on the surface of T cells, termed accessory or costimulatory molecules have been implicated in regulating the transition of a resting T cell to blast transformation, and subsequent proliferation and differentiation (T cell stimulation). Thus, in addition to the primary activation signal provided through the TCR/CD3 complex, induction of T cell responses involves a second, costimulatory signal. A costimulator of a T cell includes, but is not limited to CD28, inducible costimulatory molecule (ICOS), 4-1BB receptor (CDw137), lymphocyte function-associated antigen-1 (LFA-1), CD30, or CD154.

One such costimulatory or accessory molecule, CD28, is understood to initiate or regulate a signal transduction pathway that is distinct from those stimulated by the TCR complex. Other specific, non-limiting examples of co-stimulatory molecules are inducible costimulatory molecule (ICOS), 4-1BB receptor (CDw137), lymphocyte function-associated antigen-1 (LFA-1), CD30, or CD154 (see Salomon and Bluestone, Ann. Rev. Immunol. 19:225-52, 2001).

Thus, to induce an activated population of T cells to proliferate (i.e., a population of T cells that has received a primary activation signal) an accessory molecule on the surface of the T cell (e.g. CD28), is stimulated with a ligand which binds the accessory molecule. For example, stimulation of the accessory molecule is achieved by contacting an activated population of T cells with a ligand that binds to the accessory molecule, or with an antibody that specifically binds the accessory molecule.

In one embodiment, activation of T cells with an anti-CD3 antibody and an anti-CD28 antibody results in selective expansion of CD8⁺ T cells relative to CD4⁺ T cells. An anti-CD28 monoclonal antibody or fragment thereof capable of cross-linking the CD28 molecule, or a natural ligand for CD28 (e.g., a member of the B7 family of proteins, such as B7-1(CD80) and B7-2 (CD86) (Freedman et al. 1987. J. Immunol. 137:3260-7; Freeman et al. 1989. J. Immunol. 143:2714-22; Freeman et al. 1991. J Exp. Med. 174:625-31; Freeman et al. 1993. Science 262:909-11; Azuma et al. 1993. Nature 366:76-9; Freeman et al. 1993. J. Exp. Med. 178:2185-92) can be used to induce stimulation of the CD28 molecule. In addition, binding homologues of a natural ligand, whether native or synthesized by chemical or recombinant technique, can also be used. Ligands useful for stimulating an accessory molecule can be used in soluble form or immobilized on a solid phase surface as described herein. Anti-CD28 antibodies of fragments thereof useful in stimulating proliferation of T cells, such as CD8⁺ T cells, include monoclonal antibody 9.3, an IgG2a antibody (Dr. Jeffery Ledbetter, Bristol Myers Squibb Corporation, Seattle, Wash.), monoclonal antibody KOLT-2, an IgG1 antibody, 15E8, an IgG1 antibody, 248.23.2, an IgM antibody and EX5.3D10, an IgG2a antibody (see U.S. Pat. No. 5,858,358).

Cytokine/Interleukin (IL): A generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Many growth factors and cytokines act as cellular survival factors by preventing programmed cell death. Cytolines and interleukins include both naturally occurring peptides and variants that retain full or partial biological activity. Although specific cytokines/interleukins are described in the specification, they are not limited to the specifically disclosed peptides.

Enhance: To improve the quality, amount, or strength of something. In one embodiment, a therapy enhances the immune system if the immune system is more effective at fighting tumors. Such enhancement can be measured using any bioassay known in the art, for example, an ELISA assay.

Immobilized: Bound to a surface, such as a solid surface. A solid surface can be for example polymeric, such as polystyrene or polypropylene. In one embodiment, the solid surface is the bottom surface of a flask or a tissue culture plate. In another embodiment, the solid surface is in the form of a bead. A specific, non-limiting example of a bead is Tosylated magnetic beads (Dynal). Methods of immobilizing antibodies and peptides on a solid surface can be found in WO 94/29436, and U.S. Pat. No. 5,858,358.

Immuno-deplete: To decrease the number of immune cells, and in particular, lymphocytes, such as CD4⁺ and/or CD8⁺ cells, in a subject.

Immuno-depleting agent: One or more compounds, when administered to a subject, result in a decrease in the number of cells of the immune system (such as lymphocytes) in the subject Examples include, but are not limited to, chemotherapeutic agents, monoclonal antibodies, and other therapies disclosed in EXAMPLE 13.

Immunologically Normal: An individual that displays immune system characteristics typical for the species to which the individual belongs. These characteristics would typically include, among others, functioning B-cells and T-cells as well as structural cell components, called cell surface antigens, which act as the immunologic signature for a particular organism.

The use of such immunologically normal recipients means that an immunologically normal recipient's immune system, via its B- (humoral response) and T- (cellular response) cells, will identify the cell surface antigens of a foreign cell or an engrafted tissue as foreign. This recognition leads ultimately to an immune response against the cell or tissue, resulting in destruction of the cell or rejection of the graft. An immune response against an allogeneic tissue is known as host-versus-graft rejection. The graft can be a solid organ, such as a heart, kidney, liver, or pancreas.

Immunologically Compromised: Having reduced immune function, for example in a subject with a genotypic or a phenotypic immunodeficiency. A genotypically-immunodeficient subject has a genetic defect which results in the inability to generate either humoral or cell-mediated response. A specific, non-limiting example of a genotypically immunodeficient subject is a genotypically immunodeficient mouse, such as a SCID mouse or a bg/nu/xid mice (Andriole et al., J. Immunol. 135:2911, 1985; McCune et al., Science 241:1632, 1988). In one embodiment, a genotypically immunodeficient subject is unable to react against a foreign cell or engrafted allogeneic tissue. A phenotypically-immunodeficient subject is a subject which is genetically capable of generating an immune response, yet has been phenotypically altered such that no response is seen. In one specific, non-limiting example, a phenotypically-immunodeficient recipient is irradiated. In another specific, non-limiting example, a phenotypically-immunodeficient subject has been treated with chemotherapy.

Interferon-gamma (IFN-γ): IFN-γ includes both naturally occurring peptides, as well as IFN-γ fragments and variants that retain full or partial IFN-γ biological activity. Murine and human IFN-γ proteins have approximately 40% sequence homology. At least six different variants of naturally occurring IFN-γ have been described, and differ from each other by variable lengths of the C-terminal ends. IFN-γ is produced mainly by T-cells and natural killer cells activated by antigens, mitogens, or alloantigens. It is produced by lymphocytes expressing CD4 and CD8. The synthesis of IFN-γ is induced, among other things, by IL-2, β-FGF, and EGF.

IFN-γ can be detected by immunoassay. An ELISA test allows detection of individual cells producing IFN-γ. Minute amounts of IFN-γ can be detected indirectly by measuring IFN-induced proteins such as Mx protein. The induction of the synthesis of IP-10 has been used also to measure IFN-γ concentrations. A new bioassay employs induction of indoleamine 2,3-dioxygenase activity in 2D9 cells. A sensitive radioreceptor assay is also available.

Interleukin (IL)-2: IL-2 includes both naturally occurring and recombinant IL-2 peptides, as well as IL-2 fragments and IL-2 variants that retain full or partial IL-2 biological activity. Full-length IL-2 is a protein of 133 amino acids which does not display sequence homology to any other factors. Murine and human IL-2 are approximately 65% homologous at the protein level, and 72% at the nucleotide level in the coding region. The protein contains a single disulfide bond (positions Cys58/105) and is O-glycosylated at threonine at position 3; however non-glycosylated IL-2 is also biologically active.

IL-2 is a growth factor for all subpopulations of T-lymphocytes. It is an antigen-unspecific proliferation factor for T-cells that induces cell cycle progression in resting cells, and allows clonal expansion of activated T-lymphocytes.

IL-2 can be assayed in bioassays employing cell lines that respond to the factor (e.g., ATH8, CT6, CTLL-2, FDCPmix, HT-2, NKC-3, TALL-103). Specific ELISA assays for IL-2 and enzyme immunoassays for the soluble receptor are also available. An alternative detection method is reverse transcriptase polymerase chain reaction (RT-PCR) (Brandt et al. Lymphokine Research 5:S35-S42, 1986; Lindqvist et al. J. Immunol. Meth. 113:231-5, 1988).

IL-4: IL-4 includes both naturally occurring and recombinant IL-4 peptides, as well as IL-4 fragments and IL-4 variants that retain full or partial IL-4 biological activity. IL-4 is a protein produced mainly by a subpopulation of activated T-cells (Tc2 and Th2 cells). Full-length IL-4 is a 129 amino acid protein (20 kDa) synthesized as a precursor containing a hydrophobic secretory signal sequence of 24 amino acids. IL-4 is glycosylated at two arginine residues (positions 38 and 105) and contains six cysteine residues involved in disulfide bond formation.

IL-4 promotes the proliferation and differentiation of activated B-cells, the expression of class II MHC antigens, and of low affinity IgE receptors in resting B-cells. In addition, IL-4 enhances expression of class II MHC antigens on B-cells and can promote the B-cells' capacity to respond to other B-cell stimuli and to present antigens for T-cells.

The classical detection method for IL-4 is a B-cell costimulation assay measuring the enhanced proliferation of stimulated purified B-cells. IL-4 can be detected also in bioassays, employing IL4-responsive cells (e.g. BALM-4, BCL1, CCL-185, CT.4S, amongst others). A specific detection method for human IL-4 is the induction of CD3 in a number of B-cell lines with CD23 detected either by flow-through cytometry or by a fluorescence immunoassay.

An alternative detection method is RT-PCR (for review see: Boulay and Paul. Cur. Opin. Immunol. 4: 294-8, 1992; Paul and Ohara. Ann. Rev. Immunol. 5: 429-59, 1987). Total RNA is isolated with Trizol-LS (Life Technologies) according to manufacturer's instructions. Using the cDNA Cycle kit for RT-PCR (Invitrogen), three micrograms of each RNA are reverse transcribed into cDNA. The cDNA is quantified utilizing the CytoXpress Quantitative PCR kit for human IL-4 (Biosource International, Camarillo, Calif.).

IL-4 neutralizing agent: An agent which decreases the biological activity of IL-4, for example to an IL-4 activity level below that which can be detected using a standard immunoassay. Such agents can thus can be used to inhibit IL-4 activity. Examples of such agents, include, but are not limited to anti-IL-4 antibodies and soluble IL-4 receptor (Immunex). Particular examples include monoclonal IL-4 antibodies. Anti-human IL-4 antibodies and methods of making are known (for example see U.S. Pat. Nos. 5,863,537; 5,705,154; and 5,597,710 all to Daile et al. and U.S. Pat. No. 5,041,38 to Abrams et al.).

IL-5: IL-5 includes both naturally occurring and recombinant IL-5 peptides, as well as IL-5 fragments and IL-5 variants that retain full or partial IL-5 biological activity. Full-length murine IL-5 cDNA encodes a protein of 113 amino acids, while the human protein is 115 amino acids. Murine and human IL-5 protein sequences are approximately 70% identical. The biologically active form of IL-5 is an N-glycosylated antiparallel homodimer linked by disulfide bonds. Monomeric forms are biologically inactive. Non-glycosylated IL-5 is also biologically active.

IL-5 promotes the generation of cytotoxic T-cells from thymocytes. In thymocytes, IL-5 induces the expression of high affinity IL-2 receptors.

IL-7: IL-7 includes both naturally occurring and recombinant IL-7 peptides, as well as IL-7 fragments and IL-7 variants that retain fill or partial IL-7 biological activity. The human (152 amino acids;) and murine full-length IL-7 protein (129 amino acids) have 60% sequence homology.

IL-7 stimulates the proliferation of early and mature activated T-cells and this activity is synergised by suboptimal doses of IL-1. Unstimulated human T-cells, which also express high affinity IL-7 receptors, do not proliferate in response to IL-7. In human peripheral monocytes, IL-7 induces the synthesis of some inflammatory mediators such as IL-1, IL-6 and MIP (macrophage inflammatory protein). IL-7 also enhances the expression and secretion of IL-3 and GM-CSF in activated human T-cells. IL-7 down-regulates expression of TGF-beta in macrophages which has been suggested as an inhibitor of the antitumor immune response.

IL-7 can be assayed by its growth-promoting activity on pre-B-cells in Whitlock-Witte long-term bone marrow cultures. It can be assayed also in Bioassays employing cell lines that respond to the factor (1xN/2b; 2E8; CT6; DW34; MH11; Nb2). An alternative detection method is RT-PCR quantitation of cytokines.

IL-10: IL-10 includes both naturally occurring and recombinant IL-10 peptides, as well as IL-10 fragments and IL-10 variants that retain fill or partial IL-10 biological activity. Full-length IL-10 is a homodimeric protein with subunits having 160 amino acids. Human IL-10 shows 73% amino acid homology with murine IL-10, and 81% homology with murine IL-10 at the nucleotide level. IL-10 is produced, for example, by activated CD8+ peripheral blood T-cells and by Tc2 cells.

IL-10 can inhibit the synthesis of a number of cytolines such as IFN-γ, IL-2 and TNF-β in Tc1 subpopulations of T-cells. This activity can be antagonized by IL-4. IL-10 also inhibits mitogen- or anti-CD3-induced proliferation of T-cells in the presence of accessory cells and reduces the production of IFN-γ and IL-2.

Several methods can be used to detect IL-10, including, but not limited to: ELISA; using the murine mast cell line D36 can be used to bioassay human IL-10; and flow cytometry.

IL-12: IL-12 includes both naturally occurring and recombinant IL-12 peptides, as well as IL-12 fragments and IL-12 variants that retain full or partial IL-12 biological activity. Full-length IL-12 is a heterodimeric 70 kDa glycoprotein consisting of a 40 kDa subunit and a 35 kDa subunit linked by disulfide bonds.

IL-12 is secreted by peripheral lymphocytes after induction. It is produced mainly by B-cells and to a lesser extent by T-cells. The most powerful inducers of IL-12 are bacteria, bacterial products, and parasites. IL-12 is produced after stimulation with phorbol esters or calcium ionophore by human B-lymphoblastoid cells. IL-12 activates NK-cells positive for CD56, and this activity is blocked by antibodies specific for TNF-alpha.

IL-12 can be detected by assaying its activity as a NKSF (natural killer cell stimulatory factor), by a CLMF (cytotoxic lymphocyte maturation factor), flow cytometry, ELISA, or RT-PCR using standard methodologies and as described herein.

Isolated: An “isolated” biological component (such as a nucleic acid molecule, protein or portion of hematological material, such as blood components) has been substantially separated or purified away from other biological components in the cell of the organism in which the component naturally occurs. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins.

An isolated cell, is one which has been substantially separated or purified away from other biological components of the organism in which the cell naturally occurs. For example, an isolated Tc1 cell population is a population of Tc1 cells which is substantially separated or purified away from other blood cells, such as Tc2 cells. An isolated Tc2 cell population is a population of Tc2 cells which is substantially separated or purified away from other blood cells, such as Tc1 cells.

Lymphocytes: A type of white blood cell involved in the immune defenses of the body. There are two main types of lymphocytes: B-cells and T-cells.

Lymphoproliferation: An increase in the production of lymphocytes.

Malignant: Cells which have the properties of anaplasia invasion and metastasis.

Mammal: Includes both human and non-human mammals. Examples of mammals include, but are not limited to: primates (such as apes and chimpanzees), dogs, cats, rats, mice, cows, pigs, sheep, horses, goats, and rabbits.

Monocyte: A large white blood cell in the blood that ingests microbes or other cells and foreign particles. When a monocyte passes out of the bloodstream and enters tissues, it develops into a macrophage.

Neoplasm: Abnormal growth of cells.

Neutralizing amount: An amount of an agent sufficient to decrease the activity or amount of a substance to a level that is undetectable using standard methods.

Non-cultured Cells: Cells which have not been grown or expanded outside of the body. In one embodiment, non-cultured CD4⁺ and CD8⁺ T cells are cells that have been removed and purified from the body, but not grown in culture.

Normal Cell: Non-tumor cell, non-malignant, uninfected cell.

Purified: The term “purified” does not require absolute purity; rather, it is intended as a relative term. Thus, for example, a purified protein, nucleic acid, or cell is one in which the protein, nucleic acid, or cell is more pure than the protein, nucleic acid, or cell in its natural environment, such as within a cell or within an organism. In particular examples, substantially purified populations of cells refers to populations of cells that are at least 50%, 60%, 65%, 70%, 75%, 80%, 90%, 95%, 96%, 97%, 98% or 99% pure. In one embodiment, a substantially purified population of Tc1 cells is composed of at least about 70%, such as at least about 80%, such as at least about 90% Tc1 cells. That is, the population of Tc1 cells includes less than about 20%, such as at least about 10%, of other T lymphocytes such as Tc2 cells. In another embodiment, a substantially purified population of Tc2 cells is composed of at least about 50%, such as at least about 60%, such as at least about 70%, Tc2 cells. That is, the population of Tc2 cells includes less than about 50%, such as at least less than about 30%, of other T lymphocytes such as Tc1 cells. The purity of a Tc1 or Tc2 population can be measured based on cell surface characteristics (e.g. as measured by fluorescence activated cell sorting) or by cytoline secretion profile (e.g. as measured by an ELISA assay), as compared to a control.

Thus, in one embodiment, a population of substantially purified Tc1 cells demonstrates a 95% reduction in IL-4 secretion relative to a control Tc2 population from the same donor. In another embodiment, a substantially purified population of Tc1 cells demonstrates a 99% reduction in IL-4 secretion relative to a control Tc2 population from the same donor.

In yet another embodiment, a population of substantially purified Tc2 cells demonstrates a 95% reduction in IL-2 secretion relative to a control Tc1 population from the same donor. In another embodiment, a substantially purified population of Tc2 cells demonstrates a 99% reduction in IL-2 secretion relative to a control Tc1 population from the same donor.

One specific, non-limiting example of a substantially purified population of CD8⁺ Tc1 cells is a Tc1 population of cells that produces less than 200 pg/ml of IL-4 per 1×10⁶ CD8⁺ Tc1 lymphocytes, for example less than 100 pg/ml of IL-4 per 1×10⁶ CD8⁺ Tc1 lymphocytes, for example less than 10 pg/ml of IL-4 per 1×10⁶ CD8⁺ Tc1 lymphocytes. In further embodiments, a substantially purified population of Tc1 cells is a CD8+population of cells that produces at least 200 pg/ml of IL-2 per 1×10⁶ CD8⁺ Tc1 lymphocytes, for example at least 500 pg/ml of IL-2 per 1×10⁶ CD8⁺ Tc1 lymphocytes, for example at least 1000 pg/ml of IL-2 per 1×10⁶ CD8⁺ Tc1 lymphocytes.

One specific, non-limiting example of a substantially purified population of CD8⁺ Tc2 cells is a Tc2 population of cells that produces less than 200 pg/ml of IL-2 per 1×10⁶ CD8⁺ Tc2 lymphocytes, for example less than 100 pg/ml of IL-2 per 1×10⁶ CD8⁺ Tc2 lymphocytes, for example less than 10 pg/ml of IL-2 per 1×10⁶ CD8⁺ Tc2 lymphocytes. In further embodiments, a substantially purified population of Tc2 cells is a CD8⁺ population of cells that produces at least 200 pg/ml of IL-4 per 1×10⁶ CD8⁺ Tc2 lymphocytes, for example at least 500 pg/ml of IL-4 per 1×10⁶ CD8⁺ Tc2 lymphocytes: for example at least 1000 pg/ml of IL-4 per 1×10⁶ CD8⁺ Tc2 lymphocytes.

Reconstituting immunity: Increasing the number of lymphocytes, for example increasing the number of lymphocytes in an immuno-depleted subject, such that the immune system of the subject is enhanced relative to the immune system during immuno-depletion.

Stem Cell: A pluripotent cell that gives rise to progeny in all defined hematolymphoid lineages. In addition, limiting numbers of cells are capable of fully reconstituting a seriously immunocompromised subject in all blood cell types and their progenitors, including the pluripotent hematopoietic stem cell by cell renewal.

Subject: Includes any organism having a vascular system and hematopoietic cells in the wild-type organism. In one embodiment, the subject is a mammalian subject, such as a human or veterinary subject.

Substantially Free: Below the limit of detection for a given assay. Thus, in one specific non-limiting example, a cell culture is substantially free of IL-4 if it cannot be detected by a standard assay for analyzing IL-4 expression (e.g. below 10 pg/ml IL-4). In one embodiment, the assay is a bioassay or an ELISA assay for a specific cytokine, wherein appropriate controls are utilized to document the absence of expression of the cytokine.

Supernatant: The culture medium in which a cell is grown. The culture medium includes material from the cell, including secreted growth factors.

Therapeutically Effective Amount: An amount sufficient to achieve a desired biological effect, for example an amount that is effective to increase immunity. In particular examples, it is an amount of Tc1 and/or Tc2 cells effective to increase immunity, such as in a subject to whom it is administered, such as a subject undergoing or having a transplant, such as a stem cell transplant or an organ transplant, or a tumor. In other examples, it is an amount effective to increase immunity by more than a desired amount.

In one embodiment, the therapeutically effective amount also includes a quantity of purified Tc1 and/or Tc2 cells generated using the methods disclosed herein, sufficient to achieve a desired effect in a subject being treated. For instance, these can be an amount necessary to improve signs and/or symptoms a disease, such as a tumor or GVHD. In one embodiment, it is an amount of Tc1 and/or Tc2 cells sufficient to increase GVT and/or GVL effects in a subject relative to no administration of Tc1 and/or Tc2 cells. In another embodiment, it is an amount of Tc1 and/or Tc2 cells sufficient to decrease GVHD in a subject relative to no administration of Tc1 and/or Tc2 cells.

An effective amount of purified Tc1 and/or Tc2 cells can be administered in a single dose, or in several doses, for example daily, during a course of treatment. However, the effective amount of purified Tc1 and/or Tc2 cells will be dependent on the subject being treated, the severity and type of the condition being treated, and the manner of administration. For example, a therapeutically effective amount of purified Tc1 and/or Tc2 cells can vary from about 5×10⁶ cells per kg body weight to about 1.25×10⁸ cells per kg body weight.

The methods disclosed herein have equal application in medical and veterinary settings. Therefore, the general term “subject being treated” is understood to include all organisms (e.g. humans, apes, dogs, cats, horses, and cows) that require an increase in the desired biological effect, such as an enhanced immune response.

Therapeutically effective dose: A dose of a composition, such as cells or a drug, sufficient to improve a subject's condition. In one embodiment, it is an amount of purified Tc1 and/or Tc2 cells generated using the methods disclosed herein, sufficient to improve a subject's response to a transplant, such as an allogenic or autologous SCT. In one embodiment, it is a dose of purified Tc1 and/or Tc2 cells generated using the methods disclosed herein, sufficient to increase a GVT effect, such as a GVL effect, in a subject to whom it is administered. In a particular embodiment, it is a dose of purified Tc1 and/or Tc2 cells generated using the methods disclosed herein, sufficient to decrease a GVHD response, resulting in a regression of GVHD, or which is capable of relieving signs or symptoms caused by GVHD.

T Cell: A white blood cell critical to the immune response. T cells include, but are not limited to, CD4⁺ T cells and CD8⁺ T cells. A CD4⁺ T lymphocyte is an immune cell that carries a marker on its surface known as cluster of differentiation 4 (CD4). These cells, also known as helper T cells (Th cells), help orchestrate the immune response, including antibody responses as well as killer T cell responses. CD8⁺ T cells carry the cluster of differentiation 8 (CD8) marker. In one embodiment, CD8 T cells are cytotoxic T lymphocytes (Tc cells) which are capable of lysing target cells by direct cell contact. These cells play a role in the elimination of virus-infected cells and tumor cells, and are involved in transplant rejection processes. In another embodiment, a CD8 cell is a suppressor T cell.

T cell stimulation: A state in which a T cell response has been initiated or activated by a primary signal, such as through the TCR/CD3 complex, but not necessarily due to interaction with a protein antigen. T cell stimulation includes stimulation of a T cell with a primary signal (e.g. anti-CD3) and a co-stimulatory molecule (e.g. anti-CD28). A T cell is activated if it has received a primary signaling event that initiates an immune response by the T cell.

T cell stimulation can be accomplished, for example, by stimulating the T cell TCR/CD3 complex or via stimulation of the CD2 surface protein. An anti-CD3 monoclonal antibody can be used to activate a population of T cells via the TCR/CD3 complex. A number of anti-human CD3 monoclonal antibodies are commercially available. For example, OKT3 prepared from hybridoma cells obtained from the American Type Culture Collection (ATCC, Manassas, Va.) and monoclonal antibody G19-4 can be used to activate T cells. Similarly, binding of an anti-CD2 antibody will activate T cells.

Tc1 and Tc2 Cells: Type-1 cytotoxic T-cells (Tc1), but not type-2 cytotoxic T-cells (Tc2), are CD8⁺ T cells that secrete type 1 cytokines. Specific, non-limiting examples of type 1 cytokines are IL-2, IL-12, interferon gamma (IFN-γ), and tumor necrosis factor beta (TNF-β). Tc2 cells; but not Tc1 cells, express type 2 cytokines. Specific, non-limiting examples of type 2 cytokines are IL-4, IL-5, IL-6, and in some embodiments, IL-10.

In addition to cytokine secretion, Tc1 and Tc2 cells mediate cytolytic effects. Tc1 cells utilize both perforin- and fas-based killing pathways. Tc2 cells primarily utilize perforin-mediated cytolysis. Both Tc1 and Tc2 play a role in GVHD. Tc2 cells can reduce GVHD, and mediate a significant GVL effect.

Transformed: A transformed, transduced, or infected cell is a cell into which has been introduced a nucleic acid molecule by molecular biology techniques. As used herein, the term transformation encompasses all techniques by which a nucleic acid molecule might be introduced into such a cell, including transfection with viral vectors, transformation with plasmid vectors, and introduction of naked DNA by electroporation, lipofection, and particle gun acceleration.

Transgenic Cell: Transformed cells which contain foreign, non-native DNA.

Transplantation: The transfer of a tissue, cells, or an organ, or a portion thereof, from one subject to another subject, from one subject to another part of the same subject, or from one subject to the same part of the same subject. In one embodiment, transplantation of Tc2 cells into a subject involves obtaining blood from a donor subject, purification and generation of Tc2 cells ex vivo from the blood, and introduction of the substantially purified Tc2 cells into a recipient subject. The donor and the recipient may or may not be the same subject.

An allogeneic transplant or a heterologous transplant is transplantation from one individual to another, wherein the individuals have genes at one or more loci that are not identical in sequence in the two individuals. An allogeneic transplant can occur between two individuals of the same species, who differ genetically, or between individuals of two different species. An autologous transplant is transplantation of a tissue, cells, or a portion thereof from one location to another in the same individual, or transplantation of a tissue or a portion thereof from one individual to another, wherein the two individuals are genetically identical.

Tumor: A neoplasm. Includes solid and hematological (or liquid) tumors.

Examples of hematological tumors include, but are not limited to: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrdm's macroglobulinemia, heavy chain disease, myelodysplastic syndrome, and myelodysplasia.

Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Tumor necrosis factor beta (TNF-β): TNF-β includes both naturally occurring and recombinant TNF-β peptides, as well as TNF-β fragments and TNF-β variants that retain full or partial TNF-β biological activity. Full-length TNF-β is a protein of 171 amino acids N-glycosylated at position 62. Murine and human TNF-β are highly homologous (74%). Recombinant human proteins with deletions of 27 amino acids from the N terminus are biologically active in several bioassays.

The 5′ region of the TNF-beta promoter contains a poly(dA-dT)-rich sequence that binds the non-histone protein HMG-I which is involved in the regulation of the constitutive expression of the gene. TNF-β is produced predominantly by mitogen-stimulated T-lymphocytes and leukocytes. The synthesis of TNF-β is stimulated by interferons and IL-2.

TNF-62 can be detected in bioassays involving cell lines that respond to it (i.e. BT-20, KYM-1D4, L929, L-M, WEHI-3B). TNF-β can be detected also by an ELISA or an immunoradiometric assay (IRMA). Alternative detection methods include RT-PCR and flow cytometric quantitation.

### Method for Purifying and Expanding CD8⁺ Tc1 and Tc2 Cells

Methods of producing a population of CD8⁺ Tc1 and/or a population of Tc2 lymphocytes are provided herein.

In one embodiment, T cells are stimulated by contacting the cells with an anti-CD3 antibody and antibody that specifically binds to a T cell costimulatory molecule. In one embodiment, the antibodies are immobilized, for example, immobilized on a bead, a magnetic solid phase surface, or adhered to a tissue culture flask. T cell costimulatory molecules include, but are not limited to: CD28; inducible costimulatory molecule (ICOS); 4-1BB receptor (CDw137); lymphocyte function-associated antigen-1 (LFA-1); CD30; and CD154. Methods of stimulation of T cells with immobilized anti-CD3 and an immobilized costimulatory molecule are known (see U.S. Pat. No. 3,858,350 and PCT WO 94/29436, herein incorporated by reference in their entirety). The T cells can be stimulated once. In an alternative embodiment, the population of T cells is re-stimulated by contacting the cells with an immobilized anti-CD3 antibody and an immobilized antibody that specifically binds to a T cell costimulatory molecule. For example the re-stimulation of the T-cells can occur within about fifteen to about twenty days of the initial stimulation of the T cells.

In one embodiment, T cells are obtained or isolated from a subject. For example, T cells can be obtained from the spleen or blood of a subject.

Stimulation of the T cells is performed in the presence of a Tc1 or Tc2 supportive environment, which results in the production a population of CD8⁺ Tc1 lymphocytes which secrete at least one type I cytokine, or a population of CD8⁺ Tc2 lymphocytes which secrete at least one type II cytokine. In one embodiment, the T cells are further allowed to proliferate in the Tc1 or Tc2 supportive environment.

In one embodiment, the Tc1 supportive environment comprises at least 1 I.U./ml of IL-2, for example at least 2 I.U./ml of IL-2, for example at least 5 I.U./ml of IL-2, for example at least 10 I.U./ml of IL-2, for example at least 15 I.U./ml of IL-2, for example at least 20 I.U./ml of IL-2, and a neutralizing amount of an IL-4 neutralizing agent. Examples of IL-4 neutralizing agents that can be used to practice the methods disclosed herein, include, but are not limited to: anti-IL-4 antibodies, such as anti-IL-4 monoclonal antibodies; and soluble IL-4 receptors. In an alternative embodiment, the Tc1 supportive environment further comprises at least 1 ng/ml of IL-12, for example at least 2.5 ng/ml, such as about 2.5 ng/ml of IL-12, for example at least 10 ng/ml IL-12, for example at least or about 20 ng/ml IL-12. In another embodiment, the Tc1 supportive environment comprises about 20 I.U./ml of IL-2 and a neutralizing amount of an IL-4 neutralizing agent. In an alternative embodiment, the Tc1 supportive environment further comprises at least 1 ng/ml of IL-12, for example at least 2.5 ng/ml, such as about 2.5 ng/ml of IL-12, for example at least 10 ng/ml IL-12, for example at least or about 20 ng/ml IL-12.

Also disclosed herein are Tc2 supportive environments. In one embodiment, the Tc2 supportive environment comprises at least 100 I.U./ml of IL-2, for example at least 250 I.U./ml of IL-2, for example at least 500 I.U./ml of IL-2, for example at least 750 I.U./ml of IL-2, for example at least 800 I.U./ml of IL-2, for example at least 900 I.U./ml of IL-2, for example at least 1000 I.U./ml of IL-2, for example at least 1200 I.U./ml of IL-2, and at least 100 I.U./ml of IL-4, for example at least 250 I.U./ml of IL-4, for example at least 500 I.U./ml of IL-4, for example at least 750 I.U./ml of IL-4, for example at least 800 I.U./ml of IL-4, for example at least 900 I.U./ml of IL-4, for example at least 1000 I.U./ml of IL-4, for example at least 1200 I.U./ml of IL-4. In another embodiment, the Tc2 supportive environment comprises about 500 I.U./ml of IL-2 and about 500 I.U./ml of IL-4. In an alternative embodiment, the Tc2 supportive environment comprises about 1000 I.U./ml of IL-2 and about 1000 I.U./ml of IL-4. In another example, the Tc2 supportive environment further comprises rapamycin, such as at least 0.0001 μM, for example at least 0.001 μM, for example at least 0.01 μM, for example at least 0.1 μM, for example at least 0.5 μM, for example at least 1.0 μM, for example at least 2.0 μM, for example at least 10.0 μM of rapamycin.

In one embodiment, the purified CD8⁺ Tc1 lymphocytes secrete at least one type I cytokine. In another embodiment, the substantially purified CD8⁺ Tc1 lymphocytes are substantially free of secretion of a type II cytokine. For example, the Tc1 lymphocytes do not secrete measurable amounts of IL-4 but do secrete measurable amounts of IL-2. In a particular embodiment, the Tc1 cells secrete measurable amounts of IL-2 and/or INF-γ but not measurable amounts of IL-4. In yet another embodiment, the Tc1 cells do not secrete measurable amounts of IL-10. In a particular embodiment, the purified CD8⁺ Tc1 cells produce less than 10 pg/ml of IL-4 per 1×10⁶ CD8⁺ Tc1 lymphocytes. In yet another embodiment, the Tc1 lymphocytes produce at least 1000 pg/ml of IL-2 per 1×10⁶ CD8⁺ Tc1 lymphocytes. The secretion of cytokines can be measured using standard bioassays, such as an ELISA.

In one embodiment, the purified CD8⁺ Tc2 lymphocytes secrete at least one type II cytokine. In another embodiment, the substantially purified CD8⁺ Tc2 lymphocytes are substantially free of secretion of a type I cytokine. For example, purified CD8⁺ Tc2 lymphocytes secrete low levels of IL-2 but secrete higher amounts of IL-4. In a particular embodiment, purified CD8⁺ Tc2 lymphocytes secrete measurable amounts of IL-4 and/or IL-10. In a particular embodiment, the purified CD8⁺ Tc2 cells produce less than 10 pg/ml of IL-2 per 1×10⁶ CD8⁺ Tc2 lymphocytes. In yet another embodiment, the Tc1 lymphocytes produce at least 1000 pg/ml of IL-4 per 1×10⁶ CD8⁺ Tc2 lymphocytes. The secretion of cytokines can be measured using standard bioassays, such as an ELISA.

In one embodiment, the population of generated Tc1 or Tc2 cells is purified. In one embodiment, the purified Tc1 cells have less than 30% CD4⁺ cells, for example less than 20% CD4⁺ cells, for example less than 10% CD4⁺ cells. In another embodiment, the purified Tc2 cells have less than 50% CD4⁺ cells, for example less than 30% CD4⁺ cells. The proportion of CD4+ in the population can be measured by any means Imown to one of skill in the art. For example, fluorescence activated cell sorting can be utilized. Alternatively the supernatant content is tested for secretion of cytokines. In one embodiment, an assay, such as a bioassay, and ELISA, or a radioimmuno assay, is performed to test the cytokine secretion profile of the cells.

The methods disclosed herein can further include cryo-preserving the generated CD8⁺ Tc1 or Tc2 lymphocytes.

Also comprehended by this disclosure are CD8⁺ Tc1 and CD8⁺ Tc2 cells produced by the methods disclosed herein, as well as compositions containing such cells. In one embodiment, a substantially purified population of CD8⁺ Tc1 lymphocytes has less than 30% CD4⁺ cells, for example less than 20% CD4⁺ cells, such as less than 10% CD4⁺ cells. In another embodiment, the substantially purified population of CD8⁺ Tc1 lymphocytes produces less than about 10 pg/ml of IL-4 per 1×10⁶ CD8⁺ Tc2 lymphocytes. In yet another embodiment, the substantially purified population of CD8⁺ Tc1 lymphocytes produces at least 1000 pg/ml of IL-2 per 1×10⁶ CD8⁺ Tc1 lymphocytes.

In another embodiment, a substantially purified population of CD8⁺ Th2 lymphocytes has less than 50% CD4⁺ lymphocytes, such as less than 30% CD4⁺ lymphocytes. In another embodiment, the substantially purified population of CD8⁺ Tc2 lymphocytes produces less than about 10 pg/ml of IL-2 per 1×10⁶ CD8⁺ Tc2 lymphocytes. In yet another embodiment, the substantially purified population of CD8⁺ Tc2 lymphocytes produces at least 1000 pg/ml of IL-4 per 1×10⁶ CD8^{+ Tc}2 lymphocytes.

### Methods of Treatment by Using Purified/Expanded Tc1 and Tc2 Cells

Also disclosed herein are methods of differentially mediating transplantation responses using T cells having a specific cytokine phenotype, generated using the novel methods disclosed herein. For example, methods of reconstituting immunity in a subject having a tumor, using the Tc1 and/or Tc2 cells generated using the methods described above, are disclosed. The Tc1 and/or Tc2 cells can be administered at a dose of about 5×10⁶ to about 2×10⁸ purified CD8⁺Tc1 and/or Tc2 lymphocytes per kilogram of subject. In addition, purified populations of CD8⁺ Tc1 and/or Tc2 lymphocytes from a subject can be cryopreserved and thawed prior to administration to a recipient.

Administration of T cells which have been stimulated using an anti-CD3 monoclonal antibody and an antibody that specifically binds to a T cell costimulatory molecule (such as an anti-CD28 monoclonal antibody), in an environment which supports differentiation of type I or type II cytokine phenotype, represents a novel strategy for the regulation of GVHD and GVT, in response to a transplant, such as following a allogeneic bone marrow transplant. Stimulation of T cells allows large numbers of T cells to be generated quickly, which secrete different cytokine profiles that dictate differential in vivo function. After culture, the Tc1 and/or Tc2 cells can be cryopreserved so that they are available for therapy as needed. This treatment strategy is also of use in the treatment of tumors, such as hematological or solid tumors. Examples of hematologic or lymphoid tumors include, but are not limited to: acute lymphocytic leukemia, acute myelogenous leukemia, chronic lymphocytic leukemia, chronic myelogenous leukemia, acute myelogenous leukemia, chronic lymphocytic leukemia, chronic myelogenous leukemia, indolent non-Hodgkin's lymphoma, high-grade non-Hodgkin's lymphoma, Hodgkin's lymphoma, multiple myeloma, or myelodysplastic syndrome. Specific, non-limiting examples of solid tumors that can be treated by the method disclosed herein include, but are not limited to, breast cancer, colon cancer, ovarian cancer, renal cell carcinoma, or melanoma.

Thus, a method of transplanting immune cells to reconstitute immunity in a subject, such as a recipient having hematologic malignancy, lymphoid malignancy, or with a solid tumor is provided herein. The method includes depleting the subject's T cells that mediate graft rejection. A therapeutically effective amount of a population of cells containing CD4⁺ and CD8⁺ T cells is administered to the subject, as well as a therapeutically effective amount of a population of CD8⁺ Tc2 cells obtained using the methods disclosed herein. In one embodiment, a second dose of a therapeutically effective amount of a population of CD8⁺ Tc2 cells is administered to the subject, following administration of the first dose. In one embodiment, the population of CD8⁺ Tc2 cells is depleted of CD4⁺ cells, using standard methods. The method results in transplanting immune cells into the subject and reconstituting immunity in the subject. For an autologous transplant, the subject is both the donor and the recipient. For an allogenic transplant, the recipient subject receives the therapeutically effective amounts of a population of cells containing CD4⁺ and CD8⁺ T cells and the population of CD8⁺ Tc2 cells, from a non-self donor. The population of cells containing CD4⁺ and CD8⁺ T cells and the population of CD8⁺ Tc2 cells may or may not come from the same donor subject.

In another embodiment, the method further includes administering a therapeutically effective amount of a population of CD8⁺ Tc1 cells obtained using the methods disclosed herein to the subject. In a particular embodiment, the method includes administration of a therapeutically effective amount of a population of CD8⁺ Tc1 cells having reduced fas ligand (fasL) biological activity, since fasL is thought to contribute to GVHD, instead of the therapeutically effective amount of a population of CD8⁺ Tc2 cells. Methods of reducing fasL biological activity include, but are not limited to: subject systemic therapy with a neutralizing soluble receptor for fasL or a pharmaceutical reagent that specifically blocks fasL production.

The subject's immune system, such as T cells, can be non-selectively or selectively depleted, or ablated, by any method known in the art, for example, selective depletion or ablation of T cells or a specific subset of T cells. In one embodiment, the subject's immune system is depleted or ablated by the administration of an induction chemotherapy regimen comprising a therapeutically effective amount of etoposide, doxorubicin, vincristine, cyclophosphamide, and prednisone (EPOCH). In another embodiment, fludarabine can also be administered to improve the depletion of T cells. In yet another embodiment, fludarabine and cyclophosphamide can also be administered to improve the depletion of T cells.

Following depletion or ablation of the immune system, a therapeutically effective amount of a population of cells, including CD4⁺ and CD8⁺ T cells, are administered to the subject. In one embodiment, the cells are autologous to the subject, such as an HLA-matched donor, such as an HLA-match fist degree relative donor, or an HLA-mismatched donor. In another embodiment, the cells are allogeneic to the subject. The lymphocytes are collected by any method known to one of skill in the art. In one embodiment, the lymphocytes are collected by apheresis. In one specific non-limiting example, the lymphocyte fraction is collected by elutriation of the lymphocytes and depletion of the B cells. In another embodiment, the lymphocyte fraction is collected by elutriation and enriched for CD34⁺ cells.

Purified CD8⁺ Tc2 lymphocytes are prepared by the methods disclosed herein. Specific, non-limiting examples of a therapeutically effective amount of purified CD8⁺ Tc2 lymphocytes include purified CD8⁺ Tc2 lymphocytes administered at a dose of about 5×10⁶ cells per kilogram to about 125×10⁶ cells per kilogram, or from about 5×10⁶ cells per kilogram to about 25×10⁶ cells per kilogram, or at about 25×10⁶ cells per kilogram, or at about 125×10⁶ cells per kilogram.

The CD8⁺ Tc2 cells are administered at the same time, directly following, and/or at a time remote from the administration of the cells including CD4⁺ and CD8⁺ T cells. In one specific non-limiting example the Tc2 cells are administered within one day of the population of cells including CD4⁺ and CD8⁺ T cells. In one embodiment, the dose of cells including CD4⁺ and CD8⁺ T cells administered to the subject is from about 40×10⁶ T cells per kg to about 400×10⁶ T cells per kg. In another specific, non-limiting example, the population of cells including CD4⁺ cells and CD8⁺ cells are administered as peripheral blood stem cell transplant (PBSCT).

In addition to using Tc2 cells to treat cancer, Tc2 cells may also be used to facilitate solid organ transplantation, by decreasing the rejection of the transplanted organ. Examples of a solid organ includes, but is not limited to: lung, kidney, pancreas, heart, and liver. For example, a recipient can have a disease of end-organ failure, such as renal failure, pancreatic islet cell failure with resultant diabetes mellitus, heart failure, liver failure, or lung failure. In one embodiment, the method includes depletion of at least T cells of the recipient using any method known in the art, for example, using a chemotherapy such as the fludarabine and EPOCH regimen disclosed herein. The immuno-depletion is followed by administration of allogeneic donor CD4⁺ and CD8⁺ T cells, and donor CD8⁺ Tc2 cells (generated using the methods disclosed herein). Once donor immunity has been established in the recipient, which can be determined by donor versus host chimerism evaluation using VNTR-PCR methodology, the donor solid organ tissue is then administered to the recipient with a reduction in the occurrence of solid organ allograft rejection. Thus, the donor solid organ tissue is HLA-matched to the allogeneic donor CD4⁺ and CD8⁺ T cells and the allogeneic donor CD8⁺ Tc2 cells.

The purified populations of CD8⁺ Tc1 and Tc2 lymphocytes disclosed herein can be administered with a pharmaceutically acceptable carrier, such as saline solution. In one embodiment, compositions containing substantially purified populations of CD8⁺ Tc1 and Tc2 lymphocytes can also contain one or more therapeutic agents, such as an anti-microbial agent, for example, cyclosporin A, an anti-tumor agent, immune suppression agents such as rapamycin, and immune-depleting agents, such as a chemotherapeutic agent or a monoclonal antibody therapy. Such agents can be administered before, during or after administration of the Tc1 and/or Tc2 cells, depending on the desired effect. In one embodiment, a population of purified CD8⁺ Tc1 and/or Tc2 lymphocytes from the subject is generated prior to administration of immune-depleting agents, and the Tc1 and/or Tc2 cells administered subsequent to the administration of immune-depleting agents.

Also disclosed herein is a method for reducing GVHD in a subject. In one embodiment the method includes administering a therapeutically effective amount of purified CD8⁺ Tc1 lymphocytes prepared using the methods disclosed herein to a subject to enhance a GVT effect, wherein the Tc1 lymphocytes include a vector encoding a suicide gene. If the symptoms of GVHD appear, the subject is administered a therapeutically effective dose of a compound which is modulated into a cytotoxic effector compound by the suicide gene, thereby decreasing the population of Tc1 lymphocytes.

In another embodiment, the method includes administering to a subject having GVHD a therapeutically effective amount of a population of purified CD8⁺ Tc2 lymphocytes obtained using the methods disclosed herein, wherein administration of the population of purified CD8⁺ Tc2 lymphocytes reduces the GVHD response in the subject. In another embodiment, the method includes administering to a subject having GVHD a therapeutically effective amount of a population of purified CD8⁺ Tc1 lymphocytes generated using the methods disclosed herein, and which have reduced fasL biological activity, wherein administration of the population of purified CD8⁺ Tc1 lymphocytes having reduced fasL biological activity, reduces a GVHD response in the subject.

Also disclosed herein is a method of enhancing a graft-versus-tumor (GVT) response in a subject receiving a transplant. The method includes administering to a subject a therapeutically effective amount of a population of purified CD8⁺ Tc2 lymphocytes obtained using the methods disclosed herein, wherein administration of the Tc2 lymphocytes enhances the GVT response in the subject.

Disclosure of certain specific examples is not meant to exclude other embodiments. In addition, any treatments described in the specification are not necessarily exclusive of other treatment, but can be combined with other bioactive agents or treatment modalities.

### EXAMPLE 1

### Ex Vivo Generation of Murine CD8⁺ Tc1 and Tc2 Cells

This example describes methods used to generate murine Tc1 and Tc2 cells ex vivo. Similar methods can be used to generate Tc1 and Tc2 cells ex vivo from any subject of interest.

Spleen Harvest and T Cell Isolation

Spleens were removed aseptically from C57B1/6 mice and a single cell suspension was prepared using a syringe and petri dish containing RPMI and 10% fetal calf serum. The resulting spleen cells were enriched for T cells by negative selection by incubation with rat anti-mouse-IgG coated magnetic bioparticles which depletes B cells (Perseptive Technologies).

Ex vivo Generation of CD8⁺ Tc1 and Tc2 Cells

The freshly isolated murine splenic T cells were adjusted to a concentration of 0.3×10⁶ cells/ml and stimulated with anti-CD3/anti-CD28 coated magnetic beads (3:1 ratio of beads to T cells). The tosylated antibody coated beads (Dynal) were conjugated with anti-murine CD3 (clone 2C11; PharMingen) and with anti-murine CD28 (clone 37.51; PharMingen).

For the Tc1 culture condition, the following reagents were added at culture initiation: recombinant human IL-2 (20 I.U./ml; Chiron Therapeutics), recombinant human IL-7 (20 ng/ml; Peprotech), recombinant murine IL-12 (2.5 ng/ml; R&D Systems, Minneapolis, Minn.), a neutralizing amount of an anti-murine IL-4 monoclonal antibody (10 μg/ml; clone 11B11; obtained from the National Cancer Institute (NCI), and N-acetyl cysteine (NAC; 3.3 μM; Dey Pharmaceuticals).

Cytokine-containing media (all factors listed above except for IL-12) was added to the Tc1 culture from day 2 onward to maintain the Tc1 cells at 0.2×10⁶ cells/ml.

A “neutralizing amount” of an IL-4 neutralizing agent, such as an antibody to IL-4, is an amount required to decrease the level of recombinant human IL-4 to an undetectable level in an ELISA assay as performed using the manufacturer's instructions (IL-4 ELISA available from BioSource International, Camarillo, Calif.). The ELISA is conducted on Tc1 cell supernatants after stimulation of cells with anti-CD3, anti-CD28 beads. If the cells have 99% less IL-4 than the level of IL-4 produced by a control Tc2 cell culture (i.e. the level of IL-4 is below the 10 pg/ml detection limit for the IL-4 ELISA assay), then the cells are >99% pure for a Tc1 profile. For example, a level of IL-4 of less than 10 pg/ml (per million CD4⁺ cells for a 24 hour period of supernatant generation) demonstrates a >99% purity of Tc1 cells.

For the Tc2 culture condition, the following reagents were added at culture initiation:

recombinant human IL-2 (1000 I.U./ml), recombinant human IL-7 (20 ng/nl), recombinant murine IL-4 (1000 I.U./ml; Peprotech), and NAC (3.3 μM). Cytokine-containing media (all factors above except for IL-4) was added to the Tc2 culture from day two onward to maintain the Tc2 cells at 0.2×10⁶ cells/ml.

The median cell volume of the murine Tc1 and Tc2 cultures was determined using a Multisizer II instrument (Coulter). In successful cultures, the median cell volume for the Tc1 and Tc2 cultures increased above 500 femtoliters (fl) by day six of culture, at which time the cells were utilized for immunotherapy studies.

As shown in FIGS. 1A and 1B, CD3/CD28 stimulation in the presence of both Tc1 and Tc2 culture conditions resulted in an approximate two log expansion of T cell numbers over a five day culture period (FIG. 1A). In addition, flow cytometric analysis demonstrated that the T cells in both the Tc1 and Tc2 culture conditions were enriched for the CD8⁺ surface marker (about 79% CD8⁺ for Tc1 and about 64% CD8⁺ for Tc2 cultures) (FIG. 1B).

### EXAMPLE 2

### Cytokine Secretion Profile of Tc1 and Tc2 Cells

Tc1 and Tc2 cells were prepared as described above in EXAMPLE 1. Briefly, a splenic single cell suspension was obtained from C57B1/6 type donor mice, and depleted of B cells with rat-anti-mouse IgG bioparticles. The spleen cells were then stimulated with tosylated magnetic beads coated with rat anti-mouse CD3 and rat-anti-mouse CD28 at a bead to cell ratio of 3:1. For the Tc1 culture condition, the T cells were stimulated with the CD3, CD28 beads in media containing IL-2 (20 I.U./ml), IL-7 (20 ng/ml), IL-12 (2.5 ng/ml), neutralizing amount of antibody to IL4 (10 μg/ml and NAC (3.3 μM). For the Tc2 culture condition, the T cells were stimulated with the CD3, CD28 beads in media containing IL-2 (1000 I.U./ml), IL-7 (20 ng/ml), IL-4 (1000 I.U./ml) and NAC (3.3 μM).

On day 5 of culture, the Tc1 or Tc2 cells were harvested and brought to a final concentration of 1×10⁶ cells/ml in fresh media that contained no cytokine additives. The cells were then re-stimulated with CD3, CD28 beads at a bead to T cell ratio of 3:1. After 24 hours of stimulation, the supernatant was obtained, and cytokine content in the supernatant was determined by two-site ELISA using commercially available reagents (BioSource). Standard curves for the ELISA were determined using recombinant cytokines, and the quantity of cytokine in the experimental supernatants determined.

As FIG. 2 demonstrates, upon repeat stimulation with CD3, CD28 beads, the T cells propagated in the Tc1 culture condition produced a high level of the type I cytokines, IL-2 and IFN-γ and very low levels of the type II cytokines, IL-4 and IL-10. In contrast, the T cells propagated in the Tc2 culture condition produced a significant amount of the type II cytokines, and had an undetectable level of IL-2 and a reduced level of IFN-γ. This result demonstrates that the Tc1 culture produced a greater level of type I cytokines than the Tc2 culture. In comparison, the Tc2 culture secreted a high level of the type II cytokine, IL-4, whereas the Tc1 culture did not secrete a detectable level of IL-4. Similarly, the Tc2 culture produced an increased amount of the type II cytokine IL-10 relative to the Tc1 cells.

Therefore, using the Tc1 and Tc2 culture conditions described herein, stimulation of T cells using an anti-CD3 monoclonal antibody and antibody that specifically binds to a T cell costimulatory molecule, such as an anti-CD28 monoclonal antibody, can be utilized to generate CD8 cells of Tc1 or Tc2 cytokine phenotype. Tc1 cells are characterized by their secretion of type I cytokines, such as IL-2 and IFN-γ and their reduced level of secretion of type II cytolines, such as IL-4 and IL-10. Tc2 cells are characterized by their secretion of the type II cytolines such as IL-4 and/or IL-10, and their reduced level of secretion of the type I cytolines, such as IL-2 and/or IFN-γ.

### EXAMPLE 3

### Tc1 and Tc2 Cells Mediate Cytotoxicity by Differential Lytic Mechanisms

Murine Tc1 and Tc2 cells were prepared as described above in EXAMPLES 1 and 2. After day five in culture after initial CD3, CD28 stimulation, cells cultured under the murine Tc1 and Tc2 media were harvested and tested for their ability to lyse tumor cells using a chromium release assay, using the method disclosed in Titus et al. (J. Immunol. 138: 4018, 1987).

FIG. 3B shows the results of Tc1 and Tc2 cells against a modified Jurkat tumor cell target that allowed for evaluation of bispecific antibody-induced killing. In this assay, the Jurkat tumor cell line (Pierre Henkart, NIH) was radioactively labeled with Cr51, and subsequently chemically modified with the TNP molecule. After labeling and modifying Jurkat cells, the Tc1 or Tc2 cells were incubated with the Jurkat cells in the presence of a bispecific antibody specific for both the T cell receptor on the Tc1 or Tc2 cells and the TNP molecule present on the target cell. Therefore, this assay allowed for activation of the entire Tc1 or Tc2 cell population at the site of the tumor target, and is thus an indicator of total cytotoxic T cell potential of the Tc1 and Tc2 cells. Because this assay was performed in the presence of calcium, the killing observed is most consistent with lysis by a granule exocytosis mechanism.

In this assay, and in all of the chromium assays, four effector (T cels) to target (Jurkat cells) ratios were evaluated in the killing assay. After four hours of effector and target incubation, the supernatant was harvested and the amount of lysis determined using a gamma counter. The experimental counts per minute of radiation in the supernatant were compared with maximal and minimal lysis values obtained by target cell incubation with either sodium dodecyl sulfate (SDS) or media alone, respectively; from these values, percent specific lysis was determined. As shown in FIG. 3B, Tc2 cells have increased lysis of the target relative to Tc1 cells, using the bispecific lysis assay. Therefore, Tc2 cells have enhanced lytic function through the granule exocytosis pathway.

As shown in FIGS. 3A, 3C, and 3D, CD3, CD28-generated murine Tc1 cells have an enhanced ability to lyse tumor targets through fas/fas ligand interactions. Because calcium is required for lysis by the granule exocytosis pathway, the performance of chromium release assays in media conditions that do not allow for calcium-mediated processes reflects killing by other pathways, such as the fas pathway of cytolysis. In these experiments, to inhibit calcium-mediated processes, the assays were performed in media supplemented with MgCl₂ and EGTA.

Tc1 and Tc2 cell lysis of the L1210 tumor target, which expresses low levels of surface fas receptor, in calcium-free conditions was determined. As shown in FIG. 3A, only Tc1 cells had a nominal ability to lyse the target. When killing on the L1210 line transfected with the fas receptor was tested, it was determined that the Tc1 cells had a greatly increased ability to lyse the L1210 line (FIG. 3C). This demonstrates that Tc1 cells express functional fas ligand that can induce lysis of fas-positive tumor targets. Tc1 cell expression of fas ligand is present to a higher degree than that observed on the Tc2 population. FIG. 3D, which utilized an anti-fas blocking reagent, further confirms that the Tc1 killing in these assays was mediated through fas and fas ligand interactions.

These results demonstrate that both Tc1 and Tc2 cells mediate cytotoxicity, and are consistent with Tc1 utilization of primarily fas-based lysis and Tc2 utilization of primarily granule mediated lysis.

### EXAMPLE 4

### Generation of Human CD8⁺ Tc1 and Tc2 Cells

This example describes methods that can be used to generate Tc1 and Tc2 populations of cells from a human blood sample. However, it is understood that similar methods can be used to obtain Tc1 and Tc2 populations from other subjects and samples where lymphocytes are present.

A subject underwent a 2 to 5 liter apheresis procedure using a CS-3000 or an equivalent machine to collect lymphocytes. The apheresis product was subjected to counterflow centrifugal elutriation using standard methods. ACK lysis buffer (Biofluids, Inc., Rockville, Md.) was used to remove red blood cells from the apheresis product. The lymphocyte fraction of the elutriation product (120 to 140 fraction) was depleted of B cells by incubation with one or more mouse anti-human-B cell antibodies (for example anti-CD19, anti-CD20; anti-CD22; and/or anti-CD23; Baxter) followed by incubation with sheep anti-mouse magnetic beads (Dynal; obtained through Nexell) by standard methods using the MaxCep Device (Nexell). Cells isolated by this type of procedure have been infused into subjects without any toxicity that can be attributed to the selection procedure.

The resulting population of T cells were stimulated ex vivo using similar methods to those described in EXAMPLE 1. The T cells were plated in tissue culture flasks at a concentration of 200,000 cells per ml of culture media, comprising X-Vivo 20 media (BioWhitaker) supplemented with 5% heat-inactivated autologous plasma (herein referred to as “media”). The T cells were cultured in filtered flasks at 37° C. in 5% CO₂ humidified incubators. Magnetic beads coated with murine anti-human CD3 (clone OKT3) and murine anti-human CD28 antibodies (clone 9.3) were added to the culture at a T cell to bead ratio of 1:3. In 50 infusions of T cells grown with anti-CD3/anti-CD28 coated beads, there have been no adverse reactions except the development of an asymptomatic HAMA serologic response in one subject.

In the Tc1 culture flask, recombinant human IL-2 (20 I.U./ml), recombinant human IL-12 (2.5 ng/ml), and a neutralizing amount of an antibody to IL-4 (American Type Culture Collection (ATCC), Manassas, Va.; ATCC Number HB-9809; clone designation for this cell line is clone MP4.25D2.11) were added. A “neutralizing amount” of an IL-4 neutralizing agent, is described in EXAMPLE 1.

In the Tc2 culture flask, recombinant human IL-2 (1000 I.U./ml) and recombinant human IL-4 (1000 I.U./ml) were added. IL-4 is supplied by NCI in vials that contain 100 μg/ml per vial or 200 μg/ml per vial. The target specific activity for recombinant human IL-4 is 2.67×10⁷ I.U./mg. IL-4 can be requested by completing a Clnical Drug Request (NIH Form # 986) and mailing it to the Drug Management and Authorization Section, PMB, DCTD, NCI, 9000 Rockville Pike, EPN 707, Bethesda, Md., 20892-7422. The growth of the cells was evaluated over time. In contrast to the murine T cells described in EXAMPLES 1-3, growth of human Tc1 and Tc2 cells did not require the addition of recombinant human IL-7 or NAC.

After day 2 of culture, cells were maintained at a concentration of 0.25 to 1.0×10⁶ cells per ml by the addition of fresh media supplemented with cytolines. For Tc1 cell cultures, media was supplemented with IL-2 (1000 I.U./ml) and a neutralizing amount of antibody to IL-4. In the case of Tc2 cell cultures, media was supplemented with IL-2 (20 I.U./ml) and IL-4 (1000 I.U./ml).

The median cell volume of the Tc1 and Tc2 cultures were determined using a Multisizer II instrument (Coulter). The median cell volume of the T cells increased from 200 fl at culture initiation to over 1000 fl by one week of culture. Generally, the Tc1 and Tc2 cultures would decrease in volume to approximately 500 fl by day 15 to 20 of culture, with reduced expansion of T cell numbers observed. At that time, the cells were harvested from culture and evaluated for cytokine production and cytolytic function.

As shown in FIG. 4, CD3/CD28 stimulation in the presence of specific culture conditions resulted in expansion of both human Tc1 and Tc2 cells. The T cells cultured under Tc1 conditions had a growth expansion in the range of three logs over a 20-day culture period, and Tc2 conditions had a growth expansion in the range of five logs over a 20-day culture period. In addition, flow cytometric analysis demonstrated that the T cells in both the Tc1 and Tc2 culture conditions were enriched for the CD8⁺ surface marker (typically about 60-90%).

### EXAMPLE 5

### Cytokine Secretion Profile of Human CD8⁺ Tc1 and Tc2 Cells

Cells were prepared as described above in EXAMPLE 4. Briefly, human T cells were obtained from blood. T cells were enriched for by negative selection using mouse anti-human anti-CD-19, anti-CD20, and anti-CD-22 antibodies and sheep anti-mouse magnetic beads. T cells were plated in tissue culture flasks at a concentration of 200,000 cells per ml of culture media, comprising media. Anti-CD3, anti-CD28 coated magnetic beads were added to the culture at a T cell to bead ratio of 1:3. In the Tc1 culture flask, recombinant human IL-2 (20 I.U./ml), recombinant human IL-12 (2.5 ng/ml), and neutralizing amount of antibody to IL-4 were added. In the Tc2 culture flask, recombinant human IL-2 (1000 I.U./ml), and recombinant human IL-4 (1000 I.U./ml) were added.

Both Tc1 and Tc2 cultures were maintained at a concentration of 200,000 cells per ml by the addition of fresh media that was replete with recombinant cytokines (and anti-ILA antibody in the Tc1 culture). Cultures were monitored for cell volume by Coulter multisizer analysis, and found to increase from approximately 200 fl at culture initiation to over 1000 fl by day 7 of culture. On day 20 of culture, when the Tc1 and Tc2 culture cell volumes had returned to approximately 500 fl and the cell expansion had decreased, the cells were evaluated for cytokine production potential.

The Tc1 or Tc2 cells were harvested and restimulated with anti-CD3, anti-CD28 beads in fresh media that did not contain cytokine additives or anti-IL-4 antibody. After 24 hours, the resultant supernatant was harvested, and cytokine content of the supernatant was tested using commercially available two-site ELISA kits (BioSource). Cytokine values of the experimental samples were determined in reference to a standard curve using recombinant cytokines.

As shown in FIG. 5, T cells propagated in the Tc1 culture condition produced a high level of the type I cytokines, IL-2 and IFN-γ, and very low levels of the type II cytokines, IL-4, IL-5, and IL-10. In contrast, the T cells propagated in the Tc2 culture condition produced a significant amount of the type II cytolines, and had reduced levels of type I cytokines. This demonstrates that the Tc1 culture produced a greater level of type I cytokines than the Tc2 culture, and that the Tc2 culture secreted a high level of the type II cytokines.

Therefore, using the Tc1 and Tc2 culture conditions described herein, stimulation of human T cells using an anti-CD3 monoclonal antibody and an antibody that specifically binds to a T cell costimulatory molecule (such as an anti-CD3 monoclonal antibody), in an environment which supports differentiation of type I or type II cytokine phenotype, can be utilized to generate human CD8-enriched populations of Tc1 or Tc2 phenotype. The Tc1 cells generated by this method are characterized by their secretion of type II cytokines, such as IL-2 and IFN-γ and their reduced level of secretion of type II cytokines, such as IL-4, IL-5 and IL-10. The Tc2 cells generated by this method are characterized by their secretion of the type II cytokines such as IL-4 and/or IL-10, and their reduced level of secretion of the type I cytokines, such as IL-2 and/or IFN-γ.

### EXAMPLE 6

### Tc1 and Tc2 Cells Mediate a GVT Effect Against Breast Cancer in an Alloantigen-Driven Manner

To demonstrate that administration of Tc1 or Tc2 cells would increase survival time following administration of tumor cells, the following model was developed. In this model, a survival advantage is determined by both an ability of the Tc1/Tc2 cells to mediate an anti-tumor effect (GVL or GVT effect) and by any of negative aspects of the Tc1/Tc2 infusion (i.e. if GVHD is generated).

Host-type mice, typically F1 hybrid mice (C57B1/6×DBA/2) were lethally irradiated with total body irradiation (1100 cGy). Just after irradiation, mice received 10 million bone marrow cells from parental (C57B1/6) mice intravenously. One to two hours later, the mice were administered a tumor cell line that expresses the alloantigens present on the F1 host (from DBA/2 background). For solid tumors, mice were inoculated with 1×10⁵ TS/A breast cancer cells intravenously (i.v.). TS/A is a spontaneously occurring balb/c-derived breast cancer cell line (provided by Ron Gress, NIH). For lymphoma/leukemia models, mice were inoculated with 1×10⁵ L1210 cells i.v. (a mouse lymphocytic leukemia cell line; ATCC, Manassas, Va., catalog number CCL-119). If the recipient mouse does not receive any additional donor T cell transfers, these transplanted mice develop cancer and typically die within the first month post-transplant

To determine whether administration of additional donor T cells of Tc1 or Tc2 phenotype generated using the methods described herein can mediate an anti-tumor effect and prolong survival, the host recipient was administered T cells of an allogenic background (FIG. 6A, B6 into CB6F1 or H-2^{b} into H-2^{b/d}) or a syngeneic background (FIG. 6B, F1 into F1). Murine donor T cells were obtained and cultured under Tc1 or Tc2 culture conditions using anti-CD3, anti-CD28 co-stimulation, as described above in EXAMPLE 1. Donor Tc1 and Tc2 cells were administered to recipient mice i.v. on the same day as the irradiation and bone marrow and tumor inoculation.

As shown in FIG. 6A, mice who received the Tc 1 cells, and to a lesser extent the Tc2 cells, had a statistically significant survival advantage relative to the TSA breast cancer control group. These results demonstrate that both Tc1 and Tc2 cells can mediate a graft-versus-tumor (GVT) effect against breast cancer.

As shown in FIG. 6B, the Tc1-mediated GVT effect is not observed in the syngeneic transplant setting. Therefore, the observed Tc1- and Tc2-mediated GVT effect requires the presence of alloantigens present in the allogeneic BMT context.

Similar results were obtained using a leukemia model (L1210 cells) (see FIG. 7), indicating that Tc1 and Tc2 cells can mediate a graft-versus-leukemia (GVL) effect.

### EXAMPLE 7

### Abrogation of Tc1 Fas Ligand Reduces GVHD

As shown in EXAMPLE 6, Tc1 cells can mediate a mild GVL effect. However, recipients of Tc1 cells have also been shown to develop a moderate level of GVHD that can contribute to early lethality post-transplant. To determine if the Tc1-mediated GVHD could be reduced by abrogating the Tc1 cell fas ligand (fasL) function, a comparison was made between recipients receiving wild type (wt) or fas ligand deficient (gld) Tc1 cells.

Tc1 cells were generated from wild type (wt) or fas ligand deficient (gld) donor-type B6 (H-2^{b}) mice using the methods described in EXAMPLES 1 and 2. Recipient mice were irradiated and received an allogenic BMT (B6 into CB6F1[H-2^{b/d}]) followed by i.v. administration of 1×10⁵ L1210 cells as described in EXAMPLE 6. As shown in FIG. 7, recipients of the wt Tc1 cells had reduced survival relative to the L1210 tumor control group. Although these recipients were protected against leukemia challenge, they died of lethal GVHD. In contrast, recipients of the fasL deficient Tc1 cells had reduced GVHD and retention of some component of the GVL effect, which resulted in their improved survival relative to the tumor control group.

Therefore, the therapeutic efficacy of Tc1 cells generated using Tc1 culture conditions described herein, can be improved by methods which reduce Tc1 cell fasL, such as administration of a soluble fasL receptor.

### EXAMPLE 8

### Transfection of Suicide Gene into Tc1 Cells

Another method which can be used to obtain the benefits of the Tc1-mediated GVL effect, while decreasing the Tc1-mediated GVHD effect, is to transfect generated donor Tc1 cells with a vector encoding a suicide gene.

Suicide gene vectors encoding for the tk suicide gene are known in the art. For example, Keralavarma et al. (Blood 94(suppl.2):329b, 1999) discloses a bi-cistronic retroviral vector encoding a mutated tk suicide gene (see Black et al., Proc. Natl. Acad. Sci., 93:3525, 1996) and a non-signaling component of the human CD25 marker gene. This vector product generates a cytolytic metabolite upon the addition of ganciclovir and/or acyclovir.

The vector of Keralavarma et al. was transduced into Tc1 and/or Tc2 cells and used to delete these T cell populations as follows. Murine Tc1 and Tc2 cells were generated with CD3, CD28 co-stimulation as described above in EXAMPLES 1 and 2, in the presence of the vector. Vector was added at initiation of culture and at 24 hours. Viral supernatant and complete media ratio was 1:1. The cells were cultured in retronectin-coated flasks. After culture, the cells are incubated with a mouse anti-human CD25 reagent (PharMingen, Inc.), and then were selected using sheep anti-mouse magnetic beads (Dynal, Inc.).

As shown in FIG. 8A, in this example after six days in culture, 5.48% of the generated CD8⁺ Tc2 cells were functionally infected as measured by cell surface expression of the huCD25 antigen. The infected cells can be further purified to >90% purity using standard antibody column methods (for example see Qin et al., Proc. Natl. Acad. Sci. 98:3428-33, 2001). As shown in FIG. 8B, after a 24 hour incubation with 20 μM ganciclovir (GCV), which activates the mutated tk gene, the transfected CD8 cells were nearly completely eliminated. Similar results were obtained for Tc1 cells. Therefore, Tc1 and Tc2 cells generated using the methods disclosed herein are amenable to retroviral suicide gene infection, and furthermore that such cells can be deleted by tk modulation of a pre-metabolite.

This approach can be used for administration of allogenic T cell therapy (Tc1 or Tc2 cells) for elimination of a tumor, with subsequent treatment of the transplant recipient with GCV for control of GVHD using the methods disclosed below.

### EXAMPLE 9

### Administration of Tc2 Cells Abrogates Marrow Graft Rejection

This example describes experiments which were used to demonstrate that Tc2 cells generated using the methods disclosed herein can abrogate marrow graft rejection. Although this example describes experiments conducted in mice, it is understood that similar methods can be used in other subjects, such as humans.

Parental recipient C57B1/6 mice were irradiated with either an ablative dose (1200 cGy) or a sublethal dose (700 cGy), followed by administration of CB6F1 mice T-cell depleted bone marrow (1×10⁷ cells i.v.) one to two hours following the irradiation. T cells from donor-type F1 mice were stimulated with CD3, CD28 beads as described in EXAMPLES 1 and 2, to generate Tc1 and Tc2 populations. On the day of the transplant, usually within one to two hours following the BMT, F1 Tc1 or Tc2 cells were administered (1×10⁷ cells i.v.). On day 30 post-transplant, peripheral blood was obtained and mononuclear cells were stained with antibodies identifying host (H-2^{b}+, H-2^{d}−) versus donor (H-2^{b}+, H-2^{d}+) elements, and analyzed using flow cytometry. Antibodies were obtained from PharMingen, Inc. Two-color flow cytometry consisted of H-2 b FITC and H-2 k PE.

As shown in FIG. 9, lethal irradiation prevented marrow rejection (8/8 mice engrafting; mean of 92.8% donor chimerism). In contrast, mice receiving sublethal 700 cGy irradiation rejected the marrow graft (0/10 mice engrafting; mean of 0.2% donor chimerism). Administration of Tc1 cells partially abrogated rejection (4/8 mice engrafting; mean of 34.2% donor chimerism), whereas administration of Tc2 cells abrogated rejection in each recipient (8/8 engrafting; mean of 93.0% donor chimerism).

Therefore, Tc1 and Tc2 cells obtained using the methods disclosed herein can prevent bone marrow graft rejection by a mechanism that does not involve GVHD, although Tc2 cells are more potent in the abrogation of rejection than Tc1 cells.

### EXAMPLE 10

### Effect of Rapamycin in Tc2 Generation

To determine the effect of immune suppression agents on Tc1 and Tc2 cell generation, murine splenic CD4⁺ T cells were purified and stimulated in a Tc1 or Tc2 stimulating environment, in the presence or absence of rapamycin. One skilled in the art will understand that similar methods can be used to test other immuno-suppressive agents. In addition, using the disclosure provided in the above examples, similar methods can be used to generate human Tc1 or Tc2 cells in the presence of rapamycin.

Murine splenic CD8⁺ T cells were purified by negative selection from C57B1/6 mice, co-stimulated with anti-CD3, anti-CD28 coated magnetic beads, and cultured in the presence of cytokines culture conditions as described in EXAMPLE 1 for murine Tc1 or Tc2 cells. Tc1 and Tc2 cultures were propagated in these conditions alone, or with the additional presence of the immune suppression molecule rapamycin at 0.1 μM or 10.0 μM (Sigma, St. Louis, Mo.). Rapamycin was present from the initiation of the culture, and the cells received only the anti-3/anti-28 stimulation on day 0 (no re-stimulation). As shown in FIG. 10, rapamycin reduces CD8⁺ T-cell expansion under both Tc1 and Tc2 culture conditions. In addition, 10 μM rapamycin reduced expansion to a greater extent than 0.1 μM rapamycin.

To ensure that rapamycin does not inhibit the cytotoxic capacity of Tc2 cells (see Example 3) murine CD8⁺ T cells were isolated and stimulated with anti-CD3, anti-CD28 coated magnetic beads under Tc1 or Tc2 conditions in the presence or absence of rapamycin as described above. T cells were harvested from the various Tc2 culture conditions and evaluated in a chromium release assay for cytotoxic capacity (see EXAMPLE 3). In this assay, the effector cell was the Fc-receptor positive cell line P815 that was pre-incubated with anti-CD3 monoclonal antibody; this procedure allows Tc2 cultured effector cell triggering through the CD3 receptor at the site of the P815 tumor target. As shown in FIG. 11, the Tc2 cultured cells mediated significant lysis of the tumor target. Furthermore, culture of the Tc2 cells in either 0.1 μM or 10.0 μM rapamycin did not reduce Tc2 cell cytotoxic capacity. Only the Tc2 culture conditions are shown in FIG. 11, as the Tc1 cultured cells had nominal killing function in this assay.

To demonstrate that rapamycin does not adversely affect Tc2 cell cytokine secretion, murine CD8⁺ T cells were isolated from C57B1/6 mice and stimulated with anti-CD3, anti-CD28 coated magnetic beads under Tc2 conditions as described above, in the presence or absence of 0.1 μM or 10.0 μM rapamycin. On day six of culture, the resultant Tc2 cells were re-stimulated with anti-CD3, anti-CD28, and the 24 hour supernatant was tested for type I (IL-2, IFN-γ) and type II (IL-4, IL-5, and IL-10) cytokine content as described in EXAMPLE 2. As shown in FIG. 12, Tc2 cells cultured in rapamycin maintained a low IL-2 secretion profile, had reduced IFN-γ secretion at the high dose of rapamycin, and generally maintained type II cytokine secretion. As such, propagation of Tc2 cells in rapamycin did not abrogate, and may have enhanced, their polarity towards type II cytokine secretion.

To demonstrate that memory-type CD8⁺ T cells can be generated in the presence of rapamycin, the following methods were used. Murine CD8⁺ T cells were isolated from C57B1/6 mice and stimulated with anti-CD3, anti-CD28 coated magnetic beads under Tc2 conditions in the presence (0.1 μM or 10.0 μM) or absence of rapamycin as described above. On day six, the cultures were propagated for an additional 24 hours without further stimulation, or with anti-CD3, anti-CD28 re-stimulation with or without the continued presence of the dose of rapamycin at which the Tc2 cells were propagated. As shown in FIG. 13, culture of Tc2 cells in rapamycin resulted in a dose-dependent increase in L-selectin, CD62L. With CD3, CD28 restimulation, CD62L was rapidly down regulated; this down regulation of CD62L was only partially abrogated by continued presence of rapamycin. These results indicate that rapamycin induces Tc2 cells to obtain a memory function that would increase Tc2 cell migration and retention in the lymph node and other areas of antigen presentation. This functionality can improve the therapeutic efficacy of the Tc2 population. Such memory Tc2 cells are rapidly transformed into effector CD8 cells, as evidenced by their rapid down regulation of the CD62L.

These results indicate that rapamycin can synergize with the 3/28 methods described herein to further promote Tc2 cell generation, and that rapamycin may be a more appropriate agent to administer after an allogeneic transplantation that involves Tc2 cells, since it does not reduce Tc2-type cytolines, does it reduce the cytolytic effector function of Tc2 cells, and results in the development of a CD62L-high profile characteristic of memory CD8⁺ T cells.

### EXAMPLE 11

### General Transplant Methodology

Using the methods disclosed herein, Tc1 and/or Tc2 cells can be generated and used to enhance GVT effects, such as GVL effects, while decreasing GVHD, in allogenic transplants. In addition, Tc1 and/or Tc2 cells can be infected with a suicide vector prior to administration to a recipient, for example using the vector and methods disclosed in EXAMPLE 8, which may allow the subject to benefit from the GVT effects. If the subject later develops GVHD in response to the transplant, the transplanted Tc1 and/or Tc2 cells can be destroyed by administering to the subject a compound which is converted to a metabolite by the expressed protein product of the suicide vector, thus killing the cells causing the GVHD. Alternatively (or in addition), Tc1 cells can be generated using the methods disclosed herein, and then subject to treatment which reduces FasL biological activity.

In an allogenic transplant, donor T cells are obtained and Tc1 and/or Tc2 cells generated therefrom using the methods described herein. These cells can be cryopreserved prior to administration to a recipient. In an autologous transplant, recipient T cells are obtained prior to the transplant and cryopreserved. Tc1 and/or Tc2 cells could be generated from the T cells either prior to, or after the cryopreservation. The resulting Tc1 and/or Tc2 cells could also be cryopreserved prior to use if desired.

For subjects having a solid tumor, such as breast cancer, or a hematological tumor such as leukemia or lymphoma, an allogeneic stem cell transplant (SCT) is performed. Allogeneic SCT is usually performed from a sibling donor that matches at ⅚ or 6/6 of the HLA loci. However, only approximately 25% of cancer subjects will have that type of donor available. Therefore, it is desirable to extend allogeneic SCT to the other 75% of cancer subjects. The problem with performing HLA-mismatched SCT is that there is initially a very high rate of graft rejection, which results in failure to achieve the transplant. Because of this high rate of graft rejection, in the setting of HLA-mismatched transplantation, very high doses of chemotherapy and radiation are administered to the transplant recipient prior to the transplant. Unfortunately, this type of immuno-depleting “preparative regimen” is associated with a high mortality rate. Using the methods disclosed herein, SCT can be extended to a greater number of subjects potentially eligible for allogeneic SCT, and reduce the need for the preparative regimen or may allow for the intensity of the preparative regimen to be decreased, thus increasing the success of HLA-mismatched transplants.

Based on the results discussed above in EXAMPLE 9, the CD8⁺ Tc2 population can be used to prevent graft rejection. As such, Tc2 cells can be used to expand the transplant population to those 75% of individuals who do not have an HLA-matched sibling. Therefore, by administering Tc2 donor cells to the recipient, the PBSCT can be performed with an HLA-matched sibling or with an HLA-mismatched donor.

Once a donor is identified (either a matched sibling donor or an HLA-mismatched donor that is identified either through a registry or from a non-matched family donor, such as an haplo-identical donor—usually a parent or child of the subject), the recipient subject can receive chemotherapy or radiation as the “preparative regimen” for the transplant. After the preparative regimen, the subject receives peripheral blood stem cells from the donor. Transplantation can be performed with a drug to help prevent GVHD, such as cyclosporine A.

To further prevent GVHD, the T cells contained in the PBSCT product can be depleted using any method used by those skilled in the art, for example by CD34⁺ cell positive selection, or depletion of CD3⁺ T cells by antibody/column methodologies, thus allowing for a T cell-depleted allotransplant. However, T cell depleted allo SCT is associated with a very high rate of graft rejection, particularly in the HLA-mismatched setting. To overcome this obstacle, transplantation is performed with the addition of the ex vivo cultured Tc2 population generated using the methods disclosed herein.

### EXAMPLE 12

### Lymphocyte and PBSC Harvest and Generation of Tc1 and Tc2 Cells Prior to Transplant

This example outlines methods that can be used to obtain lymphocytes used to generate Tc1 and Tc2 cell populations, as well as methods than can be used to obtain PBSC. Such cells can be used in the treatment of subjects having a tumor, for example, by administering a therapeutically effective amount of Tc2 cells to a recipient following a SCT.

Lymphocyte Harvest

Blood is collected from a subject, such as a donor or recipient, using the method disclosed in EXAMPLE 4. The subject need not receive any particular treatment prior to harvesting the cells. Briefly, the subject undergoes a 2 to 5 liter apheresis procedure using a CS-3000 or an equivalent machine. The apheresis product is subjected to counterflow centrifugal elutriation, and the lymphocyte fraction is depleted of B cells (for example using an anti-CD-20 antibody). The resultant lymphocyte product is cryopreserved using standard methods (for example using a combination of Pentastarch and DMSO) in aliquots of 50 to 200×10⁶ cells/vial. Sterility of the population need not be tested at this stage of the Tc cell generation procedure; such testing can occur after the final co-culture of cells.

Peripheral Blood Stem Cell (PBSC) Harvest

Immediately following lymphocyte harvest, the subject receives filgrastim (G-CSF) as an outpatient (10 ug/kg/day each morning; subcutaneously) for 5, 6, or 7 days. The subject takes the filgrastim as early as possible upon awakening in the morning. This is especially important on days 5, 6, and 7 of the injections.

Apheresis typically is performed on days 5 and 6. On some occasions, sufficient numbers of CD34⁺ cells can be obtained with a single apheresis on day 5; on other occasions, apheresis is performed on days 5, 6, and 7 to reach the target CD34⁺ cell number (≧4×10⁶ per kg). The subject is instructed to take filgrastim for the complete 7 day period, unless notified by the transplant team that adequate CD34⁺ cells were harvested before day 7.

If≧3×10⁶ CD34⁺ cells per kg are harvested after apheresis on days 5, 6, and 7, no further mobilization or apheresis is performed, and the stem cell transplant can be performed with that dose of CD34⁺ cells.

In the event that less than 3×10⁶ CD34⁺ cells per kg are harvested after apheresis on days 5, 6, and 7, the subject is given two weeks of rest, and then re-treated with filgrastim followed by repeat peripheral blood stem cell harvesting.

A 15 to 25 liter large volume whole blood apheresis is performed via a 2-armed approach or via a temporary central venous catheter in the femoral position using the Baxter CS3000Plus, Cobe Spectra, or an equivalent instrument. This procedure typically takes 4 to 6 hours.

Apheresis procedure uses ACD-A anti-coagulant; alternatively, partial anti-coagulation with heparin is utilized. The apheresis product is cryopreserved and stored at −180° C. in a solution containing Plasmalyte A, Pentastarch, human serum albumin, DMSO, and preservative free heparin (10 U/ml). The concentration of CD34⁺ cells in the apheresis product is determined by flow cytometry, and the number of CD34⁺ cells in each cryopreserved bag is calculated.

If the donor and host are ABO incompatible, red blood cells are depleted from the stem cell product by standard DTM protocols.

Ex vivo Generation of CD8⁺ Tc1 and Tc2 Cells

Cryopreserved T cells are resuspended to a concentration of about 0.3×10⁶ cells per ml, and expanded using the method ex vivo, for example using the methods disclosed in EXAMPLE 4. The resulting populations of purified Tc1 and Tc2 cells can be used immediately, or cryopreserved for future use. For example, the population of purified Tc1 cells is at least 70%, 80%, 85%, or 90%, pure. If the cells have 99% less IL-4 than the level of IL-4 produced by a control Tc2 cell culture (i.e. the level of IL-4 is below the 10 pg/ml detection limit for the IL-4 ELISA assay), then the cells are >99% pure for a Tc1 profile. For example, a level of IL-4 of less than 10 pg/ml (per million CD8⁺ cells for a 24 hour period of supernatant generation) demonstrates a >99% purity of Tc1 cells.

In addition, if the T cells are tested for fungal and bacterial cultures, using standard testing done on cell products and for endotoxin content, using a limulus assay. Cell products positive for fungal, bacterial, or endotoxin content are discarded.

To estimate the number of Tc1 and Tc2 cells that could be obtained from a subject, the following calculations may be used as a guideline. About 0.5×10⁶ CD8⁺ T cells can be harvested from one ml of blood. Assuming a 2-log expansion of Tc1 and Tc2 cells in culture, it is estimated that 5×10⁷ Tc1 cells and 5×10⁷ Tc2 cells could be generated from one ml of blood. This value assumes 100% efficiency at each step of the process, which is likely not to occur; a range of 20-100% efficiency is reasonable. Therefore, about 1-5×10⁷ Tc1 cells and 1-5×10⁷ Tc2 cells could be generated per one ml of blood.

### EXAMPLE 13

### Immuno-Depletion of Transplant Recipients

In subjects receiving an allogenic SCT, or in subjects where a tumor develops following an allogenic SCT, it may be desirable or necessary to deplete the immune system and then re-build the immune system by administering a generated population of TC1 and/or Tc2 cells using the methods disclosed herein. If the transplant is an autologous transplant, the immune-depleting therapy is administered following the harvest of cell products which will be re-introduced into the subject.

Any immune-depleting method can be used. Examples include, but are not limited to immune-depleting chemotherapies and monoclonal antibody therapies.

Immune-Depleting Chemotherapies

Subjects receive at least one cycle of induction chemotherapy, even if their CD4⁺ count is less than 50 cells per μl. Placement of permanent central venous access can be performed. Ideally, steroids are not used as an anti-emetic during this chemotherapy regimen. Examples of immune depleting chemotherapy that can be used to deplete a subject's immune system prior to or following an allo-SCT include the Fludarabine/EPOCH method (Table 1) and the Fludarabine/cyclophosphamide method (fludarabine (25 mg/m² per day IV for 4 consecutive days) combined with cyclophosphamide (600 mg/m² per day IV for 4 days). However, other methods known to those skilled in the art may also be employed.

TABLE 1
Cycle 1 of Induction Chemotherapy

| Drug | Dose | Days |
|---|---|---|
| Fludarabine | 25 mg/m² per day IV | Days 1, 2, 3 |
| | Infusion over 30 minutes, | |
| | Daily for 3 days | |
| Etoposide | 50 mg/m² per day continuous IV | Days 1, 2, 3 |
| | Infusion over 24 hours, | |
| | Daily for 3 days | |
| Doxorubicin | 10 mg/m² per day continuous IV | Days 1, 2, 3 |
| | Infusion over 24 hours, | |
| | Daily for 3 days | |
| Vincristine | 0.5 mg/m² per day continuous IV | Days 1, 2, 3 |
| | Infusion over 24 hours, Daily for 3 days | |
| Cyclophosphamide | 600 mg/m² IV Infusion over 2 hr | Day 4 |
| Prednisone | 60 mg/m² per day orally, daily for 4 days | Days 1, 2, 3, 4 |
| Filgrastim | 10 ug/kg per day subcutaneously | Daily from day 5 Until |
| | | ANC > 1000/ul for two |
| | | consecutive days |

Fludarabine (Fludara, Berlex Laboratories) commercially available as FLUDARA IV. FLUDARA IV reconstituted in Sterile Water for Injection, USP, is chemically and physically stable for 24 hours at room temperature, or for 48 hours if refrigerated. Because reconstituted FLUDARA IV contains no antimicrobial preservative, care must be taken to assure the sterility of the prepared solution.

Cyclophosphamide is commercially available (Cytoxan; Mead Johnson), and can be reconstituted with sterile water for injection to a final concentration of 20 mg/ml.

Etoposide/Doxorubicin/Vincristine are all commercially available and can be admixed in 0.9% Sodium Chloride for Injection and administered as a continuous infusion.

Prednisone is commercially available in solid or liquid dosage forms.

Filgrastim (G-CSF) a commercially available recombinant human protein produced by Amgen Corp. (Thousand Oak, Calif.) marketed under the brand name Neupogen. This product differs from the natural form due to its absence of N-terminal o-glycosylation. It should not be diluted with sodium chloride solutions.

Because the primary purpose of the induction chemotherapy is to establish severe immune T cell depletion in the recipient subject prior to the SCF and administration of purified Tc1 and/or Tc2 cells (and/or additional agents such as purified, but uncultured CD4⁺ and CD8⁺ cells, or Th2 cells), the number of induction chemotherapy cycles administered is determined by the severity of immune T cell depletion observed. The CD4⁺ count can be measured by flow cytometry, for example in the interval of day 15 to day 21 of the fludarabine/EPOCH chemotherapy. If there are >50 CD4⁺ cells per μl of blood during this interval, further cycles of induction chemotherapy are administered (in an attempt to achieve greater immunosuppression prior to transplantation). If there the level of CD4⁺ cells is <50 cells per μl of blood, this indicates that the immune system of the subject is adequately depleted, and that subject receives the transplant preparative regimen.

Subjects receive the second cycle of chemotherapy on day 22 after the first cycle was initiated. However, an additional two weeks of recovery time before administration of the second cycle is provided if medically indicated (for example, for delay in neutrophil recovery, documented infection, or other complication resulting from the induction chemotherapy regimen).

If a subject develops neutropenia of less than 500 PMN's per μl for more than seven days during any cycle of induction chemotherapy, the subject receives no further induction chemotherapy. Instead, they receive a transplant preparative regimen (see below), even if the CD4⁺ count is not <50 cells per μl.

Following chemotherapy, subjects proceed to the transplant preparative regimen chemotherapy (even if the CD4⁺ count is still >50 cells per μl). If a subject develops progressive disease at any point during induction chemotherapy cycles, such a subject proceeds to the transplant preparative regimen (independent of the CD4⁺ count).

### Determination of Cycle 2 and Cycle 3 Dose Escalation

If the first cycle of induction chemotherapy does not reduce the CD4⁺ count to below 50 cells per μl and does not result in febrile neutropenia or prolonged neutropenia as evidenced by two consecutive bi-weekly ANC values less than 500 cells per μl, then the next cycle of induction chemotherapy can be dose escalated, by increasing the daily dose of fludarabine, etoposide, adriamycin, and cyclophosphamide 20%. If a third cycle of chemotherapy is required (CD4⁺ count still greater than 50) and febrile neutropenia or two timepoints of ANC less than 500 did not occur after cycle 2, then the third cycle of induction chemotherapy is administered at a further 20% escalation of doses administered for cycle 2.

### Dose Reduction of Pre-Transplant Induction Chemotherapy

In the event that more than one subject experiences a period of neutropenia (ANC less than 500 per μl) for more than 10 days, the etoposide, doxorubicin, vincristine, and prednisone is reduced from three days to two days of administration. The doses of these medications remain unchanged. In the event of this change, the cyclophosphamide and filgrastim is given on day 3. The same schedule modification described in subsection a) (above) is performed if any grade IV toxicity by the NCI Common Toxicity Criteria is observed in more than one subject.

### Transplant Preparative Regimen

On day 22 after the final cycle of induction chemotherapy, subjects are eligible to receive a transplant preparative regimen (see Table 2). Therefore, day 22 of the final induction chemotherapy cycle is transplant day −6. However, in cases where additional recovery time is required (for example, due to prolonged neutropenia, documented infection, or other medical complications of the induction regimen), an additional two weeks of recovery time is utilized prior to initiation of the transplant preparative regimen.

TABLE 2
Transplant Preparative Regimen

| Drug | Dose | Days |
|---|---|---|
| Fludarabine | 30 mg/m² per day IV Infusion | Transplant Days −6, −5, −4, −3 |
| | over 15 to 30 minutes, daily for 4 days | |
| Cyclophosphamide | 1200 mg/m² per day IV Infusion | Transplant Days −6, −5, −4, −3 |
| | over 2 hours, daily for 4 days | |
| Mesna | 1200 mg/m² per day by continuous | Transplant Days −6, −5, −4, −3 |
| | IV Infusion, daily for 4 days | |
| | (start 1 hr before cyclophosphamide)* | |

*Bag #1 of the mesna is 150 mg/m² in 250 ml over a 3 hour infusion (thus stopping when cyclophosphamide ends). Then, Mesna is given at 1200 mg/m² in 500 ml over 24 hour infusion, for four days (days −6, −5, −4, and −3).

Mesna (sodium 2-mercaptoethanesulfonate) is commercially available as Mesnex (Asta Medica). It can be diluted with normal saline to a concentration of 20 mg/ml.

### Hydration Regimen During Preparative Regimen Chemotherapy

Hydration is initiated 12 hours prior to cyclophosphamide infusion (on day −7 of the transplant). Hydration is with normal saline supplemented with 10 meq/liter KCl at a rate of 100 ml/hour. Hydration continues until 24 hours after the last cyclophoshamide dose has been completed. During hydration, 20 mg of furosemide is administered daily by IV route to maintain diuresis. If body weight in any subject increases to more than 5% above pre-cyclophosphamide weight, additional doses of furosemide are administered. In general, furosemide doses are separated by at least a four hour observation interval. During hydration, serum potassium level are monitored every 12 hours. If potassium value is >4.5 meq/l, KCl is removed from the saline infusion. If potassium value is <3.0, KCl concentration in the saline is increased to 25 meq/l. During hydration, if urine output is <1.5 ml/kg/hour, an additional 20 mg of furosemide is administered.

If administration of Tc2 cells reduced the need for the preparative regimen, the dose of cyclophosphamide could be reduced, such as reducing the dose by 50% and then observe the subject for success of engraftment.

Monoclonal Antibody Therapies

Examples of monoclonal antibody therapies that can be used to practice the disclosed methods include, but are not limited to: Rituxan and Herceptin. Rituxan is a monoclonal antibody to CD20, which is present on B cell malignancies such as lymphoma. Herceptin is a monoclonal antibody to her2-neu, which is often over-expressed on breast cancer cells. These agents are typically administered in combination with chemotherapy. In general, monoclonal-antibody based therapy is well-tolerated so a high degree of monitoring is not required.

### EXAMPLE 14

### Infection Prophylaxis

To assist in protecting a subject from infections that can result from receiving chemotherapy or other immune-depleting therapy, one or more prophylactic compounds can be administered prior to the start of the therapy, to enhance the immune system. The prophylaxis disclosed below may be administered separately, or in combination, depending on the requirements of the subject. In addition, the dosage regimens for the prophylaxis described below are known to those skilled in the art, and can be found in Mandell (Principles and Practice of Infectious Disease; 5th Edition, Copyright 2000 by Churchill Livingstone, Inc.)

For example, at the initiation of pre-transplant induction chemotherapy until administration of immunosuppressive agents is terminated, subjects may receive: trimethoprim 160 mg/sulfamethoxazole 800 mg for PCP prophylaxis (if a subject is allergic to sulfonamide antibiotics, aerosolized pentamadine (300 mg) is administered); fluconazole (oral or i.v.) for fungal and bacterial prophylaxis, and acyclovir for HSV prophylaxis.

### EXAMPLE 15

### Administration of SCT and Tc2 Cells to Recipient

GVHD Chemoprophylaxis With Cyclosporine (CSA)

CSA is initiated on the day-1 before the transplant. CSA is administered to the recipient by i.v. infusion at a dose of 2 mg/kg given over two hours. CSA is administered once every 12 hours, with each infusion administered over a 2 hour period. In the first two weeks post-transplant, CSA dose is modified to achieve adequate steady-state CSA levels. Once this intravenous dose is established and the subject is able to tolerate oral feedings (typically by day 14 post-transplant), then CSA is switched to the oral formulation. Conversion of CSA to the oral formulation is typically performed by multiplying the adequate i.v. dose by a factor of 1.5 to 2.0. Subjects are then maintained on oral CSA on a 12 hour schedule.

This dose of CSA continues until day 100 post-transplant, at which point it is gradually tapered as long as the level of GVHD is less than grade 2 (Table 3). Taper consists of a 5 to 10% dose reduction each week (subject is then taken off of CSA by day 180 post-transplant).

TABLE 3
Tapering of CSA Administration.

| | | CSA Dosage |
|---|---|---|
| Taper Step | Days post-BMT | (mg/kg/dose) |
| Taper Step 1 | 101-107 | 95% of Maintenance |
| | | Dose (M.D.) |
| Taper Step 2 | 108-114 | 90% of M.D. |
| Taper Step 3 | 115-121 | 85% of M.D. |
| Taper Step 4 | 122-128 | 80% of M.D. |
| Taper Step 5 | 129-135 | 70% of M.D. |
| Taper Step 6 | 136-142 | 60% of M.D. |
| Taper Step 7 | 143-149 | 50% of M.D. |
| Taper Step 8 | 150-156 | 40% of M.D. |
| Taper Step 9 | 157-163 | 30% of M.D. |
| Taper Step 10 | 164-170 | 20% of M.D. |
| Taper Step 11 | 171-180 | 10% of M.D. |

The decision to taper CSA before day 100 is permitted if clinically indicated. Specifically, taper of CSA before day 100 is permitted for the treatment of clinically evident progressive disease post-transplant, and for the treatment of low levels of donor chimerism post-transplant (less than 20% donor chimerism by day 60 post-transplant). Methods that can be used to determine the level of donor chimerism are described in EXAMPLE 9.

In one example, rapamycin can be used instead of, or in addition to, CSA, for GVHD prophylaxis. For example a loading dose of oral rapamycin of 15 mg per meter squared of body surface area, followed by a maintenance dose of 5 mg per meter squared per day orally for the next 13 days, can be administered (for example see Benito et al., Transplantation, 72(12):1924-29, 2001).

Peripheral Blood Stem Cell Transplantation (PBSCT)

On day 0, the recipient (who may or may not be immuno-depleted) receives the cryopreserved PBSC generated using the methods described in EXAMPLE 12. The cryopreserved PBSC product is thawed and immediately administered i.v. The target dose of the PBSC is ≧4×10⁶ CD34⁺ cells per kg. However, if donor apheresis on days 5, 6, and 7 yields a total of ≧3×10⁶ CD34⁺ cells per kg, this level of CD34+ cell dose is utilized. Usually, no steroids are allowed in the management of DMSO-related toxicities (chills, muscle aches) that may occur immediately after cellular infusion. Instead, diphenhydramine and meperidine are used.

Also on day 0 of the transplant, immediately after PBSC transfusion, recipients begin treatment with recombinant human filgrastim at a dose of 10 ug/kg/day s.c. Filgrastim continues until the ANC count is greater than 5000 cells per μl for three consecutive days.

Administration of Generated Tc2 Cells

On day 0 or day 1 of the transplant procedure, Tc2 cells are administered intravenously. Ideally, the Tc2 cells are administered within 24 hours of the PBSCT. If the Tc2 cells were previously cryopreserved, the cells are thawed and diluted in saline solution to a volume of approximately 125 to 250 ml for intravenous infusion. Tc2 cells can be administered in at least one pharmaceutically acceptable carrier, such as a saline solution. In addition, the Tc2 cells can be administered concurrently (or separately) with other therapeutic agents, such as anti-microbial agents or cyclosporine A.

In a particular example, the dose of Tc2 cells administered to a subject is in the range of: dose #1, about 5×10⁶ Tc2 cells/kg; dose #2, about 2.5×10⁷ Tc2 cells/kg; dose #3, about 1.25×10⁸ Tc2 cells/kg. Ideally, no cortico-steroids are administered in the management of DMSO-related toxicities (chills, muscle aches) that may occur immediately after cellular infusion (diphenhydiamine and meperidine are instead administered). The subject is monitored for the presence or absence of any grade 4 or 5 toxicity attributable to the Tc2 cells that occurs in the first 14 days post-transplant.

The determination of whether a Tc2 cell infusion is safe is based on the presence or absence of hyperacute GVHD and of any grade 4 or 5 toxicity attributable to the Tc2 cells that occurs in the first 14 days post-transplant. Hyperacute GVHD is a severe level of acute GVHD (grade 3 or 4) that occurs within the first 14 days post-transplant.

Toxicity is monitored by criteria established by the National Cancer Institute Cancer Therapy and Evaluation Program (NCI-CTEP). Grade 4 toxicity is considered “life-threatening” whereas Grade 5 toxicity is death. Each organ system (GI system, renal system, nervous system, etc.) is graded on the grade 0 (not observed) to grade 5 scale.

If no grade 4 or 5 toxicity attributable to the Tc2 cells is observed in an initial three subjects receiving a particular dose of Tc2 cells, then it is determined that that dose level has acceptable toxicity, and accrual to a higher dose level commences. For example, if no grade 4 or 5 toxicity attributable to the Tc2 cells is observed in an initial three subjects receiving dose #1, then it is determined that dose level #1 has acceptable toxicity, and accrual to dose level #2 commences. If grade 4 or 5 toxicity attributable to the Tc2 cells is observed in any of the initial three subjects, then accrual to dose level #1 is expanded to include a total of six subjects. If two subjects in six develop a grade IV toxicity related to the Tc2 cells, then it is determined that dose level #1 is not acceptable, and further accrual to the study stops at that point. If only one of the six subjects experiences such an adverse effect, then it is determined that dose level #1 has acceptable toxicity, and accrual proceeds to dose level #2.

Three subjects are then subjected to Tc2 cell dose level #2 (2.5×10⁷ Tc2 cells/kg). The same accrual and stopping rules apply to this dose level as those used for dose level #1. As such, either three or six subjects are accrued to dose level #2.

If it is determined that Tc2 cell dose level #2 has acceptable toxicity, accrual to the final dose level #3 starts (Tc2 cell dose of 1.2533 10⁸ cells/kg). Six subjects are evaluated on dose level #3. If more than one subject on dose level #3 develops a grade 4 or 5 toxicity attributable to the Tc2 cells, then accrual to dose level #3 stops.

The Tc2 cells disclosed herein can be administered to a subject one or more times as necessary for a particular subject. Although one infusion may be sufficient, several infusions can be performed to increase the benefit, as diseases are oftentimes chronic and difficult to treat. If multiple infusions are performed, they can be separated by a period of about four weeks. During such treatment, the subject is monitored, for example by performing tests about once or twice during each four week treatment cycle. Tests would include measurement of T cell cytolines, measurement of immune recovery panels such as T cell counts and T cell diversity and competence using methods known to those skilled in the art. In addition, tests that measure disease activity can also be performed to monitor the beneficial effect of the Tc2 cells.

Treatment of Persistent Disease Post-Transplant: DLI

After day 100 post-transplant, if a subject has persistent malignant disease, such an individual is eligible to receive an infusion of unmodified donor lymphocytes (“delayed lymphocyte infusion” or DLI) (See EXAMPLES 17 and 18). Donor lymphocytes are collected by apheresis (non-mobilized), purified by Ficoll-Hypaque procedure to a buffy coat product as per NIH DTM protocol, and administered at a therapeutically effective dose, such as about 1×10⁶ CD3⁺ T cells per kg by i.v. infusion. Alternatively (or in addition), if the Tc cells administered to the subject had a suicide vector, the suicide vector could be activated by administering a therapeutically affective amount of a compound which activates the suicide vector.

Treatment of Graft-Versus-Host Disease

In subjects where GVHD is suspected, standard clinical criteria and skin or liver biopsy information is used to establish the diagnosis. Acute GVHD is graded by the Glucksberg criteria. Subjects with clinical stage 1 or 2 of the skin without any other organ involvement are treated with a 1% hydrocortisone creme BID. In general, subjects with ≧Grade II acute GVHD are treated with high-dose corticosteroids. Subjects who fail to respond satisfactorily to corticosteroids are considered for anti-thymocyte globulin (ATG) treatment or for other experimental acute GVHD protocols, if they are available.

### EXAMPLE 16

### Modified Suicide Vectors

As an alternative method to regulate Tc1-mediated GVHD, a suicide gene can be infected into generated Tc1 cells. Several suicide genes are known in the art, for example the vector disclosed in Keralavarma et al. (Blood 94(suppl.2):329b, 1999) and discussed in EXAMPLE 8. However, the vector of Keralavarma et al. may need to be modified to allow the vector to function optimally in human cells. Therefore, to obtain a suicide vector which has clinical applicability, the following modifications can be made.

An alternative cell surface marker to CD25 can be used to enrich for (ex vivo) and track transduced T cells (in vivo). Such an alternative marker, would be a human-derived marker(s), and not endogenously expressed on the surface of human T cells generated using the methods disclosed herein. Examples of such surface markers include, but are not limited to: human CD24, truncated nerve growth factor receptor, truncated epo receptor, and B cell antigen, such as CD19, CD20 or CD22. In addition, intracellular fluorescent markers, such as green fluorescent protein, may be used.

In addition, or alternatively, the IRES elementt from the encephalomyocarditis virus can be replaced by a comparable element from a different virus. For example, an IRES element from any picornavirus genera can be used, as well as IRES elements from flaviviruses or DNA viruses such as the Kaposi's sarcoma-associated herpesvirus.

Alterations in the tk gene which still allow the gene to function as a suicide gene. For example, fragments, mutations, variations, and polymorphisims of the tk gene can be incorporated into the suicide vector which retain the ability to function as a suicide gene. Such alterations can be made using standard molecular biology methods, and the resulting vectors tested for their ability to function as a suicide vector as disclosed in EXAMPLE 8. In addition, alterations which enhance the suicide effect can also be identified. Examples of mutations in the tk gene which may be used to practice the methods disclosed herein are those mutations which result in an increased affinity or catalytic activity towards acyclovir/ganciclovir and less affinity for endogenous pools of thymine.

Other viral packaging cell lines can be used that may improve the gene transfer efficiency to the Tc1 and/or Tc2 cells. Examples, include, but are not limited to: PG13, VSV-g pseudotyped, and RD114-pseudotyped.

A suicide gene can be cloned into any vector which permits transfection or transduction into mammalian cells, such as human cells. Such vectors are well-known in the art. Examples include, but are not limited to viral vectors, such as avipox viruses, recombinant vaccinia virus, replication-deficient adenovirus strains or poliovirus, along with non-viral delivery methods using plasmid DNA.

Adenoviral vectors include essentially the complete adenoviral genome (Shenk et al., Curr. Top. Microbiol. Immunol. 11:1-39, 1984). Alternatively, the adenoviral vector is a modified adenoviral vector in which at least a portion of the adenoviral genome has been deleted. In one embodiment, the vector includes an adenoviral 5′ ITR; an adenoviral 3′ ITR; an adenoviral encapsidation signal; a DNA sequence encoding a therapeutic agent; and a promoter for expressing the DNA sequence encoding a therapeutic agent. The vector is free of at least the majority of adenoviral E1 and E3 DNA sequences, but is not necessarily free of all of the E2 and E4 DNA sequences, and DNA sequences encoding adenoviral proteins transcribed by the adenoviral major late promoter. In another embodiment, the vector is an adeno-associated virus (AAV) such as described in U.S. Pat. No. 4,797,368 (Carter et al.) and in McLaughlin et al. (J. Virol. 62:1963-73, 1988) and AAV type 4 (Chiorini et al. J. Virol. 71:6823-33, 1997) and AAV type 5 (Chiorini et al. J. Virol. 73:1309-19, 1999).

Such a vector can be constructed according to standard techniques, using a shuttle plasmid which contains, beginning at the 5′ end, an adenoviral 5′ ITR, an adenoviral encapsidation signal, and an E1a enhancer sequence; a promoter (which may be an adenoviral promoter or a foreign promoter);

a tripartite leader sequence, a multiple cloning site (which may be as herein described); a poly A signal; and a DNA segment which corresponds to a segment of the adenoviral genome. The DNA segment serves as a substrate for homologous recombination with a modified or mutated adenovirus, and may encompass, for example, a segment of the adenovirus 5′ genome no longer than from base 3329 to base 6246. The plasmid can also include a selectable marker and an origin of replication. The origin of replication may be a bacterial origin of replication. A desired DNA sequence encoding a suicide gene can be inserted into the multiple cloning site of the plasmid.

The plasmid can be used to produce an adenoviral vector by homologous recombination with a modified or mutated adenovirus in which at least the majority of the E1 and E3 adenoviral DNA sequences have been deleted. Homologous recombination can be effected through co-transfection of the plasmid vector and the modified adenovirus into a helper cell line, such as 293 cells, by CaPO4 precipitation. The homologous recombination produces a recombinant adenoviral vector which includes DNA sequences derived from the shuttle plasmid between the Not I site and the homologous recombination fragment, and DNA derived from the E1 and E3 deleted adenovirus between the homologous recombination fragment and the 3′ ITR.

In one embodiment, the adenovirus is constructed by using a yeast artificial chromosome (or YAC) containing an adenoviral genome according to the method described in Ketner et al. (Proc. Natl. Acad. Sci. USA, 91:6186-90, 1994), in conjunction with the teachings contained herein. In this embodiment, the adenovirus YAC is produced by homologous recombination in vivo between adenoviral DNA and YAC plasmid vectors carrying segments of the adenoviral left and right genomic termini. A DNA sequence encoding a therapeutic agent then is cloned into the adenoviral DNA. The modified adenoviral genome then is excised from the adenovirus YAC to be used to generate adenoviral vector particles as herein described.

In another embodiment, the viral vector is a retroviral vector. Retroviruses can be used for in vivo suicide gene expression (Orkin et al., Prog. Med. Genet.7:13042, 1988). A suicide sequence can be cloned into a retroviral vector and driven from a heterologous promoter. Examples of retroviral vectors which can be used include, but are not limited to, MMLV, spleen necrosis virus, and vectors derived from retroviruses such as RSV, Harvey Sarcoma Virus, avian leukosis virus, HIV, myeloproliferative sarcoma virus, and mammary tumor virus. The vector is generally a replication defective retrovirus particle.

Retroviral vectors are useful to effect retroviral-mediated gene transfer into eukaryotic cells. Retroviral vectors are generally constructed such that the majority of sequences coding for the structural genes of the virus are deleted and replaced by the gene(s) of interest. Most often, the structural genes (i.e., gag, pol, and env), are removed from the retroviral backbone using genetic engineering techniques known in the art. Examples include digestion with the appropriate restriction endonuclease or, in some instances, with Bal 31 exonuclease to generate fragments containing appropriate portions of the packaging signal.

Other viral transfection systems may also be utilized for this type of approach, including Vaccinia virus (Moss et al., Annu. Rev. Immunol. 5:305-24, 1987), Bovine Papilloma virus (Rasmussen et al., Methods Enzymol. 139:642-54, 1987) or members of the herpes virus group such as Epstein-Barr virus (Margolskee et al., Mol. Cell. Biol. 8:2837-47, 1988). In another embodiment RNA-DNA hybrid oligonucleotides, as described by Cole-Strauss et al. (Science 273:1386-9, 1996) are used. This technique can allow for site-specific integration of cloned sequences, permitting accurately targeted gene replacement.

Non-viral gene delivery systems can also be utilized (for example see Ledley, Curr. Opin. Biotech. 5:626-636, 1994). For example, direct injection of purified DNA into a cell or subject can be used to deliver a suicide gene. Alternatively or in addition, administration of formulations comprising DNA and lipids, proteins, peptides, and/or polymers to cells can be utilized. For example, a non-viral vector comprising a Lipofectin/integrin-targeting peptide/DNA (LID) complex can be used, as disclosed in Estruch et al. (J. Gene. Med. 3(5):488-97, 2001). In this method, the vector comprises a complex of Lipofectin and a peptide containing an integrin-targeting domain and a poly-lysine domain to which is bound plasmid DNA, containing a suicide gene.

New genes can be incorporated into proviral backbones in several ways. In the most straightforward constructions, the structural genes of the retrovirus are replaced by a single gene which then is transcribed under the control of the viral regulatory sequences within the LTR. Retroviral vectors have also been constructed which can introduce more than one gene into target cells. Usually, in such vectors one gene is under the regulatory control of the viral LTR, while the second gene is expressed either off a spliced message or is under the regulation of its own, internal promoter. Alternatively, two genes may be expressed from a single promoter by the use of an Internal Ribosome Entry Site.

The suicide gene vector is transfected into T cells generated using the methods disclosed herein. Methods of transfecting mammalian cells are well known in the art. A general strategy for transferring genes into a cell is disclosed in U.S. Pat. No. 5,529,774, incorporated by reference. Generally, a gene is cloned into a viral expression vector as described above, and that vector is then introduced into the target organism The virus infects the cells, and produces the protein sequence in vivo, where it has its desired therapeutic effect. Zabner et al. (Cell 75:207-16, 1993).

The nucleic acid sequence encoding the suicide gene is under the control of a suitable promoter. Suitable promoters which may be employed include, but are not limited to, the gene's native promoter, retroviral LTR promoter, or adenoviral promoters, such as the adenoviral major late promoter; the CMV promoter; the RSV promoter; inducible promoters, such as the MMTV promoter; the metallothionein promoter; heat shock promoters; the albumin promoter; the histone promoter; the β-actin promoter; TK promoters; B19 parvovirus promoters; tetracycline-regulatable promoters; and the ApoAI promoter. However the scope of the disclosure is not limited to specific suicide genes or promoters.

Using this approach, the suicide-gene modified Tc1 cells are administered to a subject, and the subject remains untreated until the desired anti-tumor effect occurs or until severe GVHD develops. At either of those two points, the compound that is converted to a cytolytic metabolite by the expressed protein product of the suicide gene (such as the anti-viral drug ganciclovir or acyclovir) is administered to the subject. This administration will result in death of the gene-infected cells, thus decreasing the chance of severe GVHD (such as eliminating the chance of severe GVHD) or reversing the effects of established GVHD.

### EXAMPLE 17

### Administration of Tc2 Cells in Delayed Donor Lymphocyte Infusion After Allogeneic SCT

For subjects having cancer, the development of malignant disease relapse after an allogeneic STC is a very poor prognostic sign. Recently, it has been demonstrated that with relapse after transplantation, the administration of additional donor immune cells at the time of relapse can result in tumor regressions. This form of immune therapy, because it occurs at a time remote from the original stem cell transplant procedure, is termed “delayed donor lymphocyte infusion” (DLI). Unfortunately, DLI therapy is effective in curing the cancer in only a small minority of cases. Additionally, often when DLI does result in a remission of the malignant disease, it is associated with severe GVHD that can itself be life-threatening. As such, DLI therapy has previously been limited both by poor efficacy and by toxicity.

Based on the results described above in EXAMPLE 9 and FIG. 9, Tc2 cells were capable of mediating an anti-tumor effect without significant ongoing GVHD. Therefore, the Tc2 population may be used to treat cancer with a reduced toxicity from GVHD. Tc2 cells may be administered at the time of PBSCT (as above, for the purpose of preventing graft rejection) or may be given at a time remote from the PBSCT, as an anti-cancer approach.

Tc2 cells have an application in improving the results of DLI therapy for the treatment of malignancy following an SCT, such as an allogenic SCT. For example, if following the SCT it is determined by any type of disease monitoring procedure that the subject is having progression of disease or a disease recurrence, additional CD8⁺ Tc2 cells can be administered at a therapeutically effective dose which results in an anti-tumor effect with reduced GVHD. In this Tc2-modified DLI method, the subject is immunodepleted, for example using the methods described in EXAMPLE 13, to eliminate an immune system that is not efficient in eliminating the cancer. In one embodiment, such immune-depleting chemotherapy includes fludarabine followed by EPOCH chemotherapy, with subsequent administration of fludarabine and higher doses of cyclophosphamide.

After immune depletion, the subject is administered additional donor CD4⁺ and CD8⁺ T cells in the dose range of 40 to 400×10⁶ T cells per kg. Within 24 hours after this T cell administration, the subject additionally receives ex vivo generated donor CD8⁺ Tc2 cells, using the methods disclosed herein. If desired, contaminating CD4⁺ cells can be removed from the Tc2 cell population prior to infusion of the Tc2 cell product, for example by using an immunomagnetic bead positive selection as described herein, to further enhance the Tc2 effect. This method results in a more potent DLI approach with respect to increased anti-tumor efficacy. Additionally, because the Tc2 infusion moderates GVHD, the Tc2 DLI method mediates anti-tumor effects with reduced GVHD-related toxicity.

### EXAMPLE 18

### Tc1 Cells in Delayed DLI After Allogeneic STC

Although subjects with a cancer recurrence/progression post-transplant can be treated with the Tc2 DLI strategy described in EXAMPLE 17, in some subjects, this approach may not be the most potent anti-tumor approach and further intensity of the allogeneic strategy may be required. For such subjects, a Tc1 DLI approach may be more effective, although there is a potential that the risk of GVHD would increase. Therefore, Tc2 and TC1 cells may best be utilized in a sequential fashion, with subjects that are refractory to Tc2 therapy advancing to a Tc1 infusion. Tc1 cells can be administered with pharmaceutical reagents that decrease the toxicity of TC1 cells. Examples include, but are not limited to: blockers of the TNF pathway (such as Enbrel and Infliximab) and blockers of fasL. In one embodiment, the TC1 cells are tranfected with a suicide vector.

### EXAMPLE 19

### Use of Tc2 Cells in Solid Organ Transplantation

Graft rejection remains a serious obstacle for the use of solid organ transplantation to treat end-organ failure. Host immune cells that recognize donor alloantigens present on the solid organ graft are responsible for graft rejection. Alloengraftment with reduced GVHD after Tc2 infusion represents an opportunity to transplant donor-type solid organ grafts without graft rejection.

In this method, subjects with end-organ failure are treated with immune-depleting chemotherapy as described herein. Subjects eligible for this approach include those with organ failure, such as: renal failure, heart failure, liver failure, pancreatic islet cell failure, lung failure, and those with resultant diabetes mellitus. After this immune-depleting chemotherapy, recipients receive a T cell-replete allogeneic peripheral blood stem cell transplantation from the individual who will donate the solid organ graft, or from an individual HLA-matched with the solid organ donor. Within about 24 hours of receiving the stem cell transplant, subjects then receive additional donor CD8⁺ Tc2 cells, prepared using the methods disclosed herein. Once lymphoid alloengraftment is achieved with this Tc2 approach, the recipient then receives the solid organ transplant from the donor. Alloengraftment can be tested for by using a standard VNTR-PCR methodology (Variable N terminal repeat-polymerase chain reaction) which amplifies donor versus host DNA, with readout being percent donor chimerism.

### EXAMPLE 20

### Pharmacokinetic and Immune Studies

The methods below describe how subjects can be monitored before, during, and after treatment.

Evaluation of Pre-transplant Induction Chemotherapy Cycles

Blood samples (10 cc in green-top heparinized tube) are drawn to evaluate the effects of immune depletion. This sample is drawn just prior to each cycle of induction chemotherapy (within six days of the next cycle). Experiments can include the use of flow cytometry to detect depletion of lymphoid versus myeloid subpopulations during induction chemotherapy.

Evaluation of Transplant Chemotherapy Preparative Regimen

Blood samples (10 cc in green-top heparinized tube) are drawn to evaluate the effects of the fludarabine and cyclophosphamide regimen on immune depletion in a subject. Timepoints for this aspect of the study are: 1) immediately prior to preparative regimen chemotherapy (day-6); and 2) just prior to the PBSCT (day 0). Experiments consist of flow cytometry to detect depletion of host lymphoid versus myeloid subpopulations in the peri-transplant period.

Evaluation of Type I versus Type II Cytokine Effects Post-transplant

Blood samples (30 cc in green-top heparinized tubes, and 10 cc in serum collection tubes) are drawn once weekly at the following timepoints: prior to starting induction chemotherapy, prior to each induction chemotherapy cycle, and then each week after transplant administration for the first 100 days post-transplant. Samples are delivered to the lab to perform experiments to measure plasma levels, intracellular cytokine levels, and gene expression analysis of type I versus type II cytolines in the first 100 days post-transplant.

Evaluation of Immune Reconstitution Post-Transplant

Blood (25 ml in heparinized tube) is evaluated for immune reconstitution post-transplant. Included is an evaluation of T cell receptor diversity post-transplant using a PCR-based assay. Samples are evaluated monthly for 3 months, and then every 3 months for the first two years post-transplant.

On Study Evaluation

Clinical blood tests (CBC with differential, electrolytes, liver and mineral panels): for induction chemotherapy period, day 1 and then twice per week; for inpatient period post-transplantation, daily; after discharge post-transplant, once per week. Follow-up visits are at day 140, day 180, day 290, and day 365 post-transplant. Subjects are followed every six months for one year, and then yearly until 5 years post-transplant.

Toxicity Criteria

The NCI Common Toxicity Criteria version 2.0 is used. This document can be found at the NCI website (Building, 31, Room 10A31, 31 Center Drive, MSC 2580, Bethesda, Md. 20892-2580 USA).

### EXAMPLE 21

### Pharmaceutical Compositions and Modes of Administration

Various delivery systems for administering the therapies disclosed herein are known, and include encapsulation in liposomes, microparticles, microcapsules, expression by recombinant cells, receptor-mediated endocytosis (Wu and Wu, J. Biol. Chem. 1987, 262:4429-32), and construction of therapeutic nucleic acids as part of a retroviral or other vector. Cells, such as Tc1 and Tc2 cells, are typically introduced intravenously.

The present disclosure also provides pharmaceutical compositions which include a therapeutically effective amount of purified Tc1 and/or Tc2 cells, alone or with a pharmaceutically acceptable carrier. Furthermore, the pharmaceutical compositions or methods of treatment can be administered in combination with other therapeutic treatments, such as chemotherapeutic agents and/or antibiotics, or vaccines.

Delivery Systems

The pharmaceutically acceptable carriers useful herein are conventional. Remington's Pharmaceutical Sciences, by Martin, Mack Publishing Co., Easton, Pa., 15th Edition (1975), describes compositions and formulations suitable for pharmaceutical delivery of the purified Tc1 and Tc2 cells herein disclosed. In general, the nature of the carrier will depend on the mode of administration being employed. For instance, parenteral formulations usually comprise injectable fluids that include pharmaceutically and physiologically acceptable fluids such as water, physiological saline, balanced salt solutions, aqueous dextrose, sesame oil, glycerol, ethanol, combinations thereof, or the like, as a vehicle. The carrier and composition can be sterile, and the formulation suits the mode of administration. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

The composition is typically a liquid solution, suspension, or emulsion. Embodiments of the disclosure comprising medicaments can be prepared with conventional pharmaceutically acceptable carriers, adjuvants and counterions as would be known to those of skill in the art.

The disclosure also provides a pharmaceutical pack or kit comprising one or more containers filled with one or more of the ingredients of the pharmaceutical compositions. Optionally associated with such container(s) can be a notice in the form prescribed by a governmental agency regulating the manufacture, use or sale of pharmaceuticals or biological products, which notice reflects approval by the agency of manufacture, use or sale for human administration. Instructions for use of the composition can also be included.

Such compositions are useful as therapeutic agents when constituted as pharmaceutical compositions with the appropriate carriers or diluents.

In view of the many possible embodiments to which the principles of our disclosure may be applied, it should be recognized that the illustrated embodiments are only particular examples of the disclosure and should not be taken as a limitation on the scope of the disclosure. Rather, the scope of the disclosure is in accord with the following claims. We therefore claim as our invention all that comes within the scope and spirit of these claims.

We claim:

1. A method of producing a population of CD8⁺ Tc1 lymphocytes, comprising:
stimulating a population of T cells by contacting the population of T cells with anti-CD3 monoclonal antibody and antibody that specifically binds to a T cell costimulatory molecule, in the presence of a Tc1 supportive environment, thereby producing a population of CD8⁺ Tc1 lymphocytes which secrete a type I cytokine.

2. The method of claim 1, wherein the Tc1 supportive environment comprises at least 1 IU/ml of IL-2 and a neutralizing amount of an IL-4 neutralizing agent.

3. The method of claim 2, wherein the Tc1 supportive environment comprises at least 20 IU/ml of IL-2 and a neutralizing amount of an IL-4 neutralizing agent.

4. The method of claim 2, wherein the Tc1 supportive environment further comprises at least 1 ng/ml of IL-12.

5. The method of claim 4, wherein the TC1 supportive environment further comprises about 2.5 ng/ml of IL-12.

6. The method of claim 1, further comprising allowing the stimulated population of T cells to proliferate in the Tc1 supportive environment

7. The method of claim 6, wherein the Tc1 supportive environment comprises at least 1 IU/ml of IL-2 and a neutralizing amount of an IL-4 neutralizing agent.

8. The method of claim 7, wherein the Tc1 supportive environment comprises about 20 IU/ml of IL-2 and a neutralizing amount of an IL-4 neutralizing agent.

9. The method of claim 1, wherein the population of T cells are obtained from a subject.

10. The method of claim 1, wherein the type I cytokine is IL-2 or IFN-γ.

11. The method of claim 10, wherein the type I cytokine is IL-2.

12. The method of claim 1, wherein the population of CD8⁺ Tc1 lymphocytes is purified.

13. The method of claim 12, wherein the population of purified CD8⁺ Tc1 lymphocytes comprises less than 20% CD4⁺ lymphocytes.

14. The method of claim 1, wherein the population of CD8⁺ Tc1 lymphocytes produces less than 10 pg/ml of IL-4 per 1×10⁶ CD8⁺ Tc1 lymphocytes.

15. The method of claim 1, wherein the population of CD8⁺ Tc1 lymphocytes produces at least 1000 pg/ml of IL-2 per 1×10⁶ CD8⁺ Tc1 lymphocytes.

16. The method of claim 1, further comprising re-stimulating the CD8⁺ Tc1 lymphocytes with an immobilized anti-CD3 monoclonal antibody and an immobilized antibody that specifically binds to a T cell costimulatory molecule, after allowing the cells to proliferate in the TC1 supportive environment.

17. The method of claim 1, wherein the IL-4 neutralizing agent is an anti-IL-4 antibody.

18. The method of claim 1, wherein the antibody that specifically binds to a T cell costimulatory receptor specifically binds CD28, inducible costimulatory molecule (ICOS), 4-1BB receptor (CDw137), lymphocyte function-associated antigen-1(LFA-1), CD30, or CD154.

19. The method of claim 18, wherein the antibody that specifically binds a T cell costimulatory molecule specifically binds CD28.

20. The method of claim 1, wherein the antibodies are immobilized.

21. The method of claim 20, wherein the immobilized anti-CD3 monoclonal antibody and the immobilized antibody that specifically binds a T cell costimulatory molecule are immobilized on a magnetic solid phase surface.

22. A CD8⁺ Tc1 cell produced by the method of claim 1.

23. A method of producing a population of CD8⁺ Tc1 lymphocytes, comprising:
stimulating a population of T cells obtained from a subject by contacting the population with an immobilized anti-CD3 monoclonal antibody and an immobilized antibody that specifically binds to a T cell costimulatory molecule in the presence of a first Tc1 supportive environment, wherein the first Tc1 supportive environment comprises about 20 IU/ml of IL-2, about 2.5 ng/ml IL-12, and a neutralizing amount of an IL-4 neutralizing agent, thereby forming a stimulated population of CD8⁺ T cells; and
allowing the stimulated population of CD8⁺ T cells to proliferate in a second Tc1 supportive environment comprising about 1000 IU/ml of IL-2, and a neutralizing amount of an IL-4 neutralizing agent; thereby producing a population of CD8⁺ TC1 lymphocytes, wherein the population of CD8⁺ Tc1 lymphocytes secrete a type-I cytokine.

24. A method of producing a population of substantially purified CD8⁺ Tc1 lymphocytes, comprising:
stimulating a population of T cells obtained from a subject by contacting the population with an immobilized anti-CD3 monoclonal antibody and an immobilized anti-CD28 monoclonal antibody in the presence of a first Tc1 supportive environment, wherein the first Tc1 supportive environment comprises about 20 IU/ml of IL-2, about 2.5 ng/ml IL-12, and a neutralizing amount of an IL-4 neutralizing agent, thereby forming a stimulated population of CD8⁺ T cells;
allowing the stimulated population of CD8⁺ T cells to proliferate in a second Tc1 supportive environment comprising about 1000 IU/ml of IL-2, and a neutralizing amount of an IL-4 neutralizing agent; and
re-stimulating the T lymphocytes in a media that does not contain IL-2 or IL-12 additives, thereby producing a population of CD8⁺ Tc1 lymphocytes, wherein the population of CD8⁺ Tc1 lymphocytes secrete a type-I cytokine.

25. The method of claim 24, wherein the first Tc1 supportive environment further comprises about 2.5 ng/ml IL-12.

26. The method of claim 24, further comprising cryo-preserving the purified CD8⁺ Tc1 lymphocytes.

27. A substantially purified population of CD8⁺ Tc1 lymphocytes, wherein the population comprises less than about 30% CD4⁺ lymphocytes.

28. The substantially purified population of CD8⁺ Tc1 lymphocytes of claim 27 wherein the population comprises less than about 10% CD4⁺ lymphocytes.

29. The substantially purified population of CD8⁺ Tc1 lymphocytes of claim 27 wherein the population comprises less than about 30% Tc2 lymphocytes.

30. The substantially purified population of CD8⁺ Tc1 lymphocytes of claim 27, wherein the population produces less than about 10 pg/ml of IL-4 per 1×10⁶ CD8⁺ Tc1 lymphocytes.

31. The substantially purified population of CD8⁺ Tc1 lymphocytes of claim 27, wherein the population produces at least 1000 pg/ml of IL-2 per 1×10⁶ CD8⁺ Tc1 lymphocytes.

32. A method of producing a population of CD8⁺ Tc2 lymphocytes, comprising:
stimulating a population of T cells by contacting the population of T cells with anti-CD3 monoclonal antibody and antibody that specifically binds to a T cell costimulatory molecule, in the presence of a Tc2 supportive environment, thereby producing a population of CD8⁺ Tc2 lymphocytes which secrete a type II cytokine.

33. The method of claim 32, wherein the Tc2 supportive environment comprises at least 500 IU/ml of IL-2 and at least 500 IU/ml of IL-4.

34. The method of claim 33, wherein the Tc1 supportive environment comprises at least 1000 IU/ml of IL-2 and at least 1000 IU/ml of IL-4.

35. The method of claim 32, further comprising allowing the stimulated population of T cells to proliferate in the Tc2 supportive environment.

36. The method of claim 32, wherein the population of T cells are obtained from a subject.

37. The method of claim 32, wherein the type II cytokine is IL-4 or IL-10.

38. The method of claim 37, wherein the type II cytokine is IL-4.

39. The method of claim 32, wherein the population of CD8⁺ Tc2 lymphocytes comprises less than 50% CD4⁺ lymphocytes.

40. The method of claim 32, wherein the population of CD8⁺ Tc2 lymphocytes produces less than 10 pg/ml of IL-2 per 1×10⁶ CD8⁺ Tc2 lymphocytes.

41. The method of claim 32, wherein the population of CD8⁺ Tc2 lymphocytes produces at least 1000 pg/ml of IL-4 per 1×10⁶ CD8⁺ Tc2 lymphocytes.

42. The method of claim 32, further comprising re-stimulating the CD8⁺ Tc2 lymphocytes with an immobilized anti-CD3 monoclonal antibody and an immobilized antibody that specifically binds to a T cell costimulatory molecule, after allowing the cells to proliferate in the Tc2 supportive environment

43. The method of claim 32, wherein the antibody that specifically binds to a T cell costimulatory receptor specifically binds CD28, inducible costimulatory molecule (ICOS), 4-1BB receptor (CDw137), lymphocyte function-associated antigen-1(LFA-1), CD30, or CD154.

44. The method of claim 43, wherein the antibody that specifically binds a T cell costimulatory molecule specifically binds CD28.

45. The method of claim 32, wherein the antibodies are immobilized.

46. The method of claim 45, wherein the immobilized anti-CD3 monoclonal antibody and the immobilized antibody that specifically binds a T cell costimulatory molecule are immobilized on a magnetic solid phase surface.

47. A CD8⁺ Tc2 cell produced by the method of claim 32.

48. A method of producing a population of CD8⁺ Tc2 lymphocytes, comprising:
stimulating a population of T cells obtained from a subject by contacting the population with an immobilized anti-CD3 monoclonal antibody and an immobilized antibody that specifically binds to a T cell costimulatory molecule in the presence of a Tc2 supportive environment, wherein the Tc2 supportive environment comprises about 1000 IU/ml of IL-2, and about 1000 IU/ml of IL-4, thereby forming a stimulated population of T cells; and
allowing the stimulated population of CD8⁺ T cells to proliferate in the Tc2 supportive environment, thereby producing a population of CD8⁺ Tc2 lymphocytes, wherein the population of CD8⁺ Tc2 lymphocytes secrete a type II cytokine.

49. A method of producing a population of substantially purified CD8⁺ Tc2 lymphocytes, comprising:
stimulating a population of T lymphocytes from a subject by contacting the population with an immobilized anti-CD3 monoclonal antibody and an immobilized anti-CD28 monoclonal antibody in the presence of a Tc2 supportive environment comprising about 1000 IU/ml of IL-2 and about 1000 IU/ml of IL-4;
allowing the stimulated population of CD8⁺ T cells to proliferate in the Tc2 supportive environment; and
re-stimulating the T lymphocytes in an environment that does not contain IL-2 or IL-4 additives, thereby producing a population of substantially purified CD8⁺ Tc2 lymphocytes.

50. The method of claim 49, further comprising cryo-preserving the purified CD8⁺ Tc2 lymphocytes.

51. A substantially purified population of CD8⁺ Tc2 lymphocytes, wherein the population comprises less than about 50% CD4⁺ lymphocytes.

52. The substantially purified population of CD8⁺ Tc2 lymphocytes of claim 51 wherein the population comprises less than about 30% CD4⁺ lymphocytes.

53. The method of claim 33, wherein the Tc2 supportive environment further comprises about 0.1 μM to about 10 μM rapamycin.

54. The substantially purified population of CD8⁺ Tc2 lymphocytes of claim 51, wherein the population produces less than about 10 pg/ml of IL-2 per 1×10⁶ CD8⁺ Tc2 lymphocytes.

55. The substantially purified population of CD8⁺ Tc2 lymphocytes of claim 51, wherein the population produces at least 1000 pg/ml of IL-4 per 1×10⁶ CD8⁺ Tc2 lymphocytes.

56. A method of transplanting immune cells to reconstitute immunity in a subject having a tumor, comprising:
immuno-depleting at least T cells in the subject;
administering to the subject a therapeutically effective amount of a population of cells comprising CD4⁺ and CD8⁺ T cells; and
administering to the subject a therapeutically effective amount of a population of purified CD8⁺ Tc2 lymphocytes obtained using the method of claim 32, thereby transplanting immune cells into the subject and reconstituting immunity in the subject.

57. The method of claim 56, wherein the population of cells comprising CD4⁺ and CD8⁺ T cells are administered as a peripheral blood stem cell product.

58. The method of claim 56, wherein the tumor is a hematologic malignancy, a lymphoid malignancy, or a solid tumor.

59. The method of claim 58, wherein the solid tumor is a carcinoma.

60. The method of claim 59, wherein the solid tumor is a renal cell carcinoma, ovarian cancer, breast cancer, colon cancer or malignant melanoma.

61. The method of claim 58, wherein the hematologic or lymphoid malignancy is acute lymphocytic leukemia, acute mylogenous leukemia, chronic lymphocytic leukemia, chronic myelogenous leukemia, acute myelogenous leukemia, chronic lymphocytic leukemia, chronic myelogenous leukemia, indolent non-Hodgkin's lymphoma, high-grade non-Hodgkin's lymphoma, Hodgkin's lymphoma, multiple myeloma, or myelodysplastic syndrome.

62. The method of claim 56, wherein the population of cells comprising CD4⁺ and CD8⁺ T cells and the population of purified CD8⁺ Tc2 lymphocytes are allogenic to the subject

63. The method of claim 56, wherein immuno-depleting at least T cells in the subject comprises administering to the subject an induction chemotherapy regimen comprising a therapeutically effective amount of etoposide, doxorubicin, vincristine, cyclophosphamide, and prednisone.

64. The method of claim 63, wherein the induction chemotherapy regimen further comprises administering to the subject a therapeutically effective amount of fludarabine.

65. The method of claim 63, wherein immuno-depleting at least T cells in the subject further comprises administering to the subject a transplant preparative chemotherapy regimen comprising a therapeutically effective amount of fludarbine and cyclophosphamide.

66. The method of claim 56, wherein the population of cells comprising CD4⁺ and CD8⁺ T cells are from an HLA-matched first degree relative donor.

67. The method of claim 56, wherein the population of cells comprising CD4⁺ and CD8⁺ T cells are from an HLA-mismatched donor.

68. The method of claim 56, wherein the administration of the population of cells comprising CD4⁺ and CD8⁺ T cells, and the population of purified CD8⁺ Tc2 lymphocytes, is simultaneous.

69. The method of claim 56, wherein the population of purified CD8⁺ Tc2 lymphocytes, are administered following the administration of the population of cells comprising CD4+and CD8+T cells.

70. The method of claim 69, wherein the administration of the population of purified CD8⁺ Tc2 lymphocytes is within one day of the administration of the population of cells comprising CD4⁺ and CD8⁺ T cells.

71. The method of claim 56, wherein the population of purified CD8⁺ Tc2 lymphocytes are administered at a time remote from the administration of the population of cells comprising CD4⁺ and CD 8⁺ T cells.

72. The method of claim 56, further comprising:
administering to the subject another therapeutically effective amount of a population of purified CD8⁺ Tc2 lymphocytes obtained using the method of claim 32, thereby transplanting immune cells into the subject and reconstituting immunity in the subject.

73. The method of claim 56, further comprising:
administering to the subject another therapeutically effective amount of a population of purified CD8⁺ Tc2 lymphocytes obtained using the method of claim 32, wherein CD4⁺ cells have been depleted from the population of purified CD8⁺ Tc2 lymphocytes, thereby transplanting immune cells into the subject and reconstituting immunity in the subject.

74. The method of claim 56, further comprising:
administering to the subject a therapeutically effective amount of a population of purified CD8⁺ Tc1 lymphocytes obtained using the method of claim 1, thereby transplanting immune cells into the subject and reconstituting immunity in the subject.

75. The method of claim 56, wherein the population of purified CD8⁺ Tc2 lymphocytes are administered at a dose of about 5×10⁶ cells per kilogram to about 125×10⁶ cells per kilogram.

76. The method of claim 56, wherein the population of cells comprising CD4⁺ and CD8⁺ T cells are administered at a dose of about 40×10⁶ T cells per kilogram to about 400×10⁶ T cells per kilogram.

77. A method of decreasing rejection of a solid organ in a recipient, comprising:
immuno-depleting at least T cells in the recipient;
administering to the recipient a therapeutically effective amount of donor allogeneic peripheral blood cells comprising stem cells, CD4⁺ cells, and CD8⁺ cells; and
administering to the recipient a therapeutically effective amount of donor CD8⁺ Tc2 lymphocytes prepared using the method of claim 32;
transplanting a solid organ into the recipient, wherein the solid organ is HLA-matched to the donor CD8⁺ Tc2 lymphocytes and to the donor allogeneic peripheral blood cells, wherein administration of the allogeneic donor peripheral blood cells and the allogeneic CD8⁺ Tc2 lymphocytes results in immune reconstitution of the subject, thereby decreasing rejection of the solid organ.

78. The method of claim 77, wherein the solid organ is a kidney, liver, heart, lung or pancreas.

79. The method of claim 77, wherein the recipient has a disorder selected from the group consisting of renal failure, kidney failure, heart failure, liver failure, lung failure and diabetes.

80. The method of claim 77, wherein the solid organ, the donor CD8⁺ Tc2 lymphocytes and the donor allogeneic peripheral blood cells are from the same donor.

81. A method of transplanting immune cells to reconstitute immunity in a subject having a tumor, comprising:
obtaining a population of purified CD8⁺ Tc1 lymphocytes using the method of claim 1;
reducing a fas ligand (FasL) biological activity in the population of purified CD8⁺ Tc1 lymphocytes;
immuno-depleting at least T cells in the subject;
administering to the subject a therapeutically effective amount of a population of cells comprising CD4⁺ and CD8⁺ T cells; and
administering to the subject a therapeutically effective amount of the population of purified CD8⁺ Tc1 lymphocytes having reduced FasL biological activity, thereby transplanting immune cells into the subject and reconstituting immunity in the subject.

82. A method of reducing graft-versus-host disease (GVHD) in a subject, comprising:
obtaining a population of purified CD8⁺ Tc1 lymphocytes using the method of claim 1;
infecting the population of purified CD8⁺ Tc1 lymphocytes with a vector encoding a suicide gene;
administering to the subject a therapeutically effective amount of the population of purified CD8⁺ Tc1 lymphocytes infected with the vector encoding the suicide gene, thereby enhancing a graft-versus-tumor effect; and
administering to the subject a therapeutically effective amount of a compound that is converted to a cytolytic metabolite by an expressed protein product of the suicide gene, thereby decreasing the population of CD8⁺ Tc1 lymphocytes administered to the subject thereby reducing GVHD in the subject.

83. A method of reducing a GVHD response in a subject having GVHD, comprising:
administering to the subject a therapeutically effective amount of a population of purified CD8⁺ Tc2 lymphocytes obtained using the method of claim 32, wherein administration of the population of purified CD8⁺ Tc2 lymphocytes reduces the GVHD response in the subject.

84. A method of reducing a GVHD response in a subject having GVHb, comprising:
obtaining a population of purified CD8⁺ Tc1 lymphocytes using the method of claim 1;
reducing a FasL biological activity in the population of purified CD8⁺ Tc1 lymphocytes; and
administering to the subject a therapeutically effective amount of the population of purified CD8⁺ Tc1 lymphocytes having reduced FasL biological activity, wherein administration of the population of purified CD8⁺ Tc1 lymphocytes having reduced FasL biological activity reduces the GVHD response in the subject.

85. A method of enhancing a graft-versus-tumor (GVT) response in subject having a transplant, comprising:
administering to a subject a therapeutically effective amount of a population of purified CD8⁺ Tc2 lymphocytes obtained using the method of claim 32, wherein administration of the population of purified CD8⁺ Tc2 lymphocytes enhances the GVT response in the subject.

86. A viral suicide vector comprising:
a mutated thymidine kinase cDNA sequence which enhances a suicide effect;
an internal ribosome entry site; and
a human cell surface antigen not endogenously expressed on a surface of human Tc1 or Tc2 cells.

87. A method of reducing graft-versus-host disease (GVHD) in a subject, comprising:
obtaining a population of purified CD8⁺ Tc1 lymphocytes using the method of claim 1;
infecting the population of purified CD8⁺ Tc1 lymphocytes with a vector encoding the suicide gene of claim 86;
administering to the subject a therapeutically effective amount of the population of purified CD8⁺ Tc1 lymphocytes infected with the vector encoding the suicide gene, thereby enhancing a graft-versus-tumor effect; and
administering to the subject a therapeutically effective amount of a compound that is converted to a cytolytic metabolite by an expressed protein product of the suicide gene, thereby decreasing the population of CD8⁺ Tc1 lymphocytes administered to the subject thereby reducing GVHD in the subject.
